(19) United States
(12) Patent Application Publication
(10) Pub. No.: US 2024/0217917 A1
(43) Pub. Date: Jul. 4, 2024

(54) PHOTOLYTICALLY DEGRADABLE COMPOUNDS AND METHODS

(71) Applicant: The University of Stavanger, Stavanger (NO)

(72) Inventors: Magne Olav SYDNES, Stavanger (NO); Leiv Kristen SYDNES, Blomsterdalen (NO); Vebjørn EIKEMO, Stavanger (NO)

(73) Assignee: The University of Stavanger, Stavanger (NO)

(21) Appl. No.: 18/556,435

(22) PCT Filed: Apr. 13, 2022
(86) PCT No.: PCT/EP2022/059863

§ 371 Date: Oct. 20, 2023

(30) Foreign Application Priority Data
Apr. 20, 2021 (SE) 2150492-3

(51) Int. Cl. C07C 213/02; C07D 205/04

(52) U.S. Cl. CPC C07C 213/02; C07D 205/04

(57) ABSTRACT

The present invention relates to a method of synthesizing a photolytically degradable compound which method comprises identifying a biologically active target molecule comprising substituted aromatic group(s): providing a starting material, which presents at least the aromatic group(s) of the target molecule and optionally an equal chain length to the target molecule: and synthesizing a photolytically degradable compound by introducing one or more substitutions in the starting material. The target molecule may e.g. be an antimicrobial molecule, an antibacterial molecule, or a pesticide molecule. The photolytically degradable compounds according to the invention may comprise at least one C—N, C—S or C—O bond and optionally at least one benzylic C—C bond capable of heterolytic cleavage if exposed to light at basic pH in aqueous environment.

The present invention relates to the field of organic chemistry, and more specifically to a method of designing and preparing biologically active molecules, which undergo degradation easily in the environment. More specifically, the invention encompasses strategic design of molecules that presents a biological function of interest, but in addition also comprise a functionality which enables a subsequent photolytic decomposition under ambient conditions. Thus, the invention provides biologically active molecules which may first be used e.g. as parasiticides, pesticides, antimicrobials, such as antibacterials, or drugs, such as antibiotics, and subsequently easily be incapacitated by exposure to light.

### BACKGROUND

Resistant bacteria strains are emerging due to overuse of antibiotics in human medicine and agriculture. Multi-drug resistant (MDR) bacteria are already a widespread problem and several public-health organisations have described the situation as a crisis that would have catastrophic consequences. Further, alarming data from around the world has shown that over 100 pharmaceutical compounds have been detected in drinking water, wastewater, ground water, and marine organisms. One example is the antibiotic ciprofloxacin that has been detected in wastewater across the world. Conventional UV treatment of effluent water containing the compound has been carried out but proved to have no effect. Another example is the antibiotic chloramphenicol, a broad-spectrum antibiotic.

The aquaculture industry is a large global business, which is estimated to continue growing. For example, in Norway, aquaculture products are the largest export commodity after oil and gas, and this makes it important to solve problems associated with its production. One such problem is the salmon lice (Lepeophtheirus salmonis), feeding on skin, mucus, and blood from the host, which may also induce epizootics in wild fish. Since the salmon farms are commonly using open-net pens, such lice infestations can easily move to adjacent farms and may also infect the local populations of fishes, such as sea trout (Salmo trutta) and wild Atlantic salmon (Salmo salar).

In order to handle the problems related to salmon lice infestation, large quantities of pesticides, such as diflubenzuron and teflubenzuron, are currently being applied:

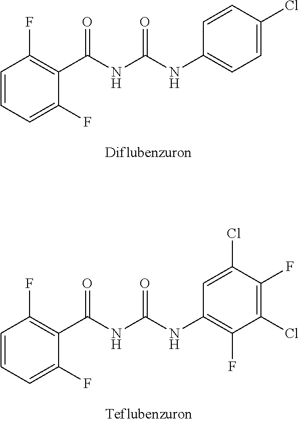 [SMILES: O=C(NC(=O)c1c(F)cccc1F)Nc1ccc(Cl)cc1 ; O=C(NC(=O)c1c(F)cccc1F)Nc1cc(Cl)c(F)c(Cl)c1F]

These compounds act as chitin synthase inhibitors, meaning that the lice are unable to form the chitin-rich exoskeleton after moulting ultimately leading to their death.

However, there are many studies showing that these medicines are persistent and stable with a stipulated half-life of 170 days after excretion by the salmon. They have been detected in water as far away as 1100 m from pens and have proven to exhibit negative impacts on king crab, shrimp, squat lobster, and European and Norway lobster. In fact, if moulting was imminent, the detected levels of teflubenzuron were sufficient to induce mortality. Additionally, the maximum-residue level (MRL) value in saithe and the crustaceans mentioned above exceeded that of Atlantic salmon, meaning that the food safety also has to be considered. Thus, there is an urgent need to generate chitin synthase inhibitors that has a shorter half-life.

Light-degradable compounds have been described, such as CA2972079 (Jayraman et al.), which decomposes in the environment where strong selective pressures imposed by antibiotic residuals are known to accelerate antibiotic resistance. Phosphopyricin is described as an antimicrobial compound with a photosensitive chemical architecture that would reduce accumulation in the environment.

Despite the above, in order to protect the environment from harmful biologically active compounds, there is still a need for overall strategies which create efficient ways of eliminating residues of pharmaceuticals and other biologically active compunds in all aqueous compartments.

### SUMMARY OF THE INVENTION

A first aspect of the invention is a method of synthesizing photolytically degradable compounds as defined in claim 1. The invention also encompasses compounds synthesized according to the claimed methods. More specifically, the invention relates to a method of synthesizing a photolytically degradable compound, which method comprises the steps of
a) identifying a biologically active target molecule comprising one or more substituted aromatic group(s);
b) providing a starting material which presents at least one aromatic group identical to said one or more substituted aromatic group(s); and
c) synthesizing the photolytically degradable compound by introducing a nitro substitution in said aromatic group of the starting material;

wherein the photolytically degradable compound is defined by the general Formula I:

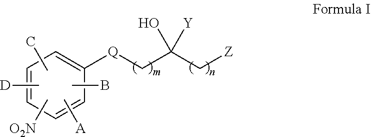

wherein
at least one of A and B and C and D is
   —CN,
   —CH₂NR¹R² or —[CH₂NR¹R²R³]⁺ wherein R¹, R² and R³ are each selected from a group consisting of H and branched or unbranched C₁-C₆ alkyl;
   O(CH₂)ₒ(CX₂)ₚ(CH₂)_{q}(CHX)ᵣ(CH₂)ₛ(CX₂)ₜ(CH₂)ᵤ(CX₃)ᵥ(CHX₂)_{w}(CH₂X)ₓ(CH₃)_{y} where
   o=0-5, p=0-5, q=0-5, r=0-5, s=0-4, t=0-4, u=0-4, v=0-1, w=0-1, x=0-1, y=0-1, provided that in each compound, only one of v, w, x, and y are 1 while the other three are 0, and
where X is F and/or Cl,

while the other three of A, B, C and D are
1) each independently selected from the group consisting of H, F, Cl, I, Br, CN, CF₃, C₁-C₆ alkyl, alkoxy with a C₁-C₆ alkyl, NH₂, NHR¹, NR¹R², and [NR¹R²R³]⁺ where R¹, R² and R³ are as defined above, and
2) can be randomly attached to the four remaining carbon atoms in the phenyl ring available for substitution;

Q is either
NH, NR⁴, [NR⁴R⁵]⁺, NOH, NOR⁴, NNH₂, NNHR⁴, [NNHR⁴R⁵]+, NNR⁴R⁵, and [NNR⁴R⁵R⁶]⁺ where R⁴, R⁵, and R⁶ are a branched or unbranched C₁-C₃ alkyl;
or O;
or S, [SR⁴]⁺, SO and SO₂;

m is 0-4, such as 1-4;

Y is selected from a group consisting of H and branched or unbranched C₁-C₆ alkyl;

n is 0-4;

Z is either
H, or
NHC(O)R⁷
   wherein R⁷ is either R⁵, or monohalogenated-perhalogenated derivatives of R⁵,
      wherein the halogen is F and/or Cl;
aryl; such as

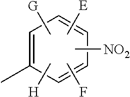

which aryl may be substituted with a nitro group, and wherein
substituents E and F and G and H are defined as above for A, B, C and D, respectively.

Another second aspect of the invention is a method of reducing the load of antimicrobial molecules in waste- or drinking water, wherein water including an antibacterial molecule obtained and synthesized according to the method of the invention that is subjected to light to photolytically degrade.

A further aspect of the invention is a method of controlling salmon lice in aquaculture, wherein a pesticide obtained and synthesized according to the method of the invention is used to treat salmon in aquaculture.

Further embodiments, details and advantages will appear from the dependent claims as well as from the detailed disclosure and experimental part that follows below.

### BRIEF DESCRIPTION OF THE DRAWINGS

FIG. 1: ¹H-NMR spectra before and after photolysis of compound 1 at pH 7, 9, 11, and 13;

FIG. 2: ¹H-NMR spectra before and after photolysis of compound 2 at pH 9, 11, and 13;

FIG. 3: ¹H-NMR spectra before and after photolysis of compound 3 at pH 9, 11, and 13;

FIG. 4: ¹H-NMR spectra before and after photolysis of compound 4 at pH 9, 11, and 13;

FIG. 5: ¹H-NMR spectra before and after photolysis of compound 5 at pH 9, 11, and 13,

FIG. 6. ¹H-NMR spectra of (A) compound 6 before irradiation, (B) decomposition product, and (C) after photodegradation;

FIG. 7: UV-vis spectra of compounds 1-5;

FIG. 8. UV-vis spectra of compounds 6-9; and

FIG. 9 a-c. Minimum inhibitory concentration (MIC) plots for Compound 6 (•), Compound 7 (▴), Compound 8 (▪), and Compound 9 (♦).

### DETAILED DESCRIPTION OF THE INVENTION

As will appear from below, the present invention provides a method of designing and preparing biologically active molecules, such as antimicrobial or pesticide molecules, which undergoes degradation easily in aqueous environments, hence denoted photolytically degradable compounds. The biologically active molecules comprise substituted aromatic groups.

The biological activity may be any activity selected from a broad range of activities, such as a capability of destroying, reducing or otherwise harm a microbial or other function, such as the biological function of a microbe, parasite, or the like.

A first aspect of the invention is a method of synthesizing a photolytically degradable compound with biological activity resembling a compound with a known biological activity, such as an antimicrobial, antibacterial, or pesticide compound.

Specifically, a first aspect of the invention is a method of synthesizing photolytically degradable compounds, which method comprises the steps of
a) identifying a biologically active target molecule comprising one or more substituted aromatic group(s);
b) providing a starting material which presents at least one aromatic group identical to said one or more substituted aromatic group(s); and
c) synthesizing the photolytically degradable compound by introducing a nitro substitution in said aromatic group of the starting material;

wherein the photolytically degradable compound is defined by the general Formula I:

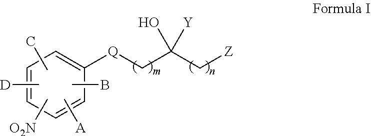

wherein
at least one of A and B and C and D is
   —CN,
   —CH₂NR¹R² or —[CH₂NR¹R²R³]⁺ wherein R¹, R² and R³ are each selected from a group consisting of H and branched or unbranched C₁-C₆ alkyl;
   O(CH₂)ₒ(CX₂)ₚ(CH₂)_{q}(CHX)ᵣ(CH₂)ₛ(CX₂)ₜ(CH₂)ᵤ(CX₃)ᵥ(CHX₂)_{w}(CH₂X)ₓ(CH₃)_{y} where
   o=0-5, p=0-5, q=0-5, r=0-5, s=0-4, t=0-4, u=0-4, v=0-1, w=0-1, x=0-1, y=0-1, provided that in each compound, only one of v, w, x, and y are 1 while the other three are 0, andy are 1 while the other three are 0, and
   where X is F and/or Cl,

while the other three of A, B, C and D are
1) each independently selected from the group consisting of H, F, Cl, I, Br, CN, CF3, C₁-C₆ alkyl, alkoxy with a C₁-C₆ alkyl, NH₂, NHR¹, NR¹R², and [NR¹R²R³]⁺ where R¹, R² and R³ are as defined above, and
2) can be randomly attached to the four remaining carbon atoms in the phenyl ring available for substitution;

Q is either
NH, NR⁴, [NR⁴R⁵]⁺, NOH, NOR⁴, NNH₂, NNHR⁴, [NNHR⁴R⁵]⁺, NNR⁴R⁵, and [NNR⁴R⁵R⁶]⁺ where R⁴, R⁵, and R⁶ are a branched or unbranched C₁-C₃ alkyl;
or O;
or S, [SR⁴]⁺, SO and SO₂;

m is 0-4, such as 1-4;

Y is selected from a group consisting of H and branched or unbranched C₁-C₆ alkyl;

n is 0-4;

Z is either
H, or
NHC(O)R⁷
   wherein R⁷ is either R⁵, or monohalogenated-perhalogenated derivatives of R⁵,
      wherein the halogen is F and/or Cl;
aryl; such as

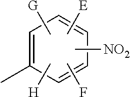

which aryl may be substituted with a nitro group,
and wherein
substituents E and F and G and H are defined as above for A, B, C and D, respectively.

In step c), the substitution may be a meta-nitro substitution; a para-nitro substitution; or a meta-nitro substitution and a para-nitro substitution is introduced in said aromatic group of the starting material.

Further, the Z group in Formula I may be aryl; such as

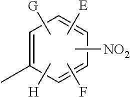

wherein
   the nitro group is in meta-position or para-position to the carbon attaching the Z group to the carbon chain of Formula (I); and
   substituents E and F and G and H are as defined above for A, B, C and D.

More specifically, the Z group in Formula I may be beta lactam.

Further, in Formula I, X may be F and/or Cl, such as F.

In this context, the term “identifying” a target molecules is understood to be the determination by the user of the present method to decide which molecule the photolytically degradable compound synthesized according to the invention should resemble, i.e. to choose a suitable target molecule. The synthesis of the invention may be a selective synthesis.

In one embodiment, the target molecule is selected from groups of antimicrobial molecules and pesticides. In one embodiment the target molecule is an antibacterial molecule.

The skilled person will be able to decide the most advantageous parameters for the synthesis according to the invention, such as concentrations, volumes and temperatures, taking into consideration the relevant target molecule and if needed, by performing simple test runs. For example, the present method may be performed at room temperature, but for some target molecules, higher temperatures may be more advantageous, such as temperatures above 30° C., such as above 40° C. or above 50° C. in an advantageous embodiment, the synthesis is performed at a temperature of about 60° C.

In brief, photolytically degradable compounds synthesized according to the invention may comprise both a C—C bond and a C—N, C—S or C—O bond which are capable of being cleaved in a photo-retro-aldol reaction via abstraction of a proton, see e.g. Scheme 1 below (compound 1 can be found in Table 1 below).

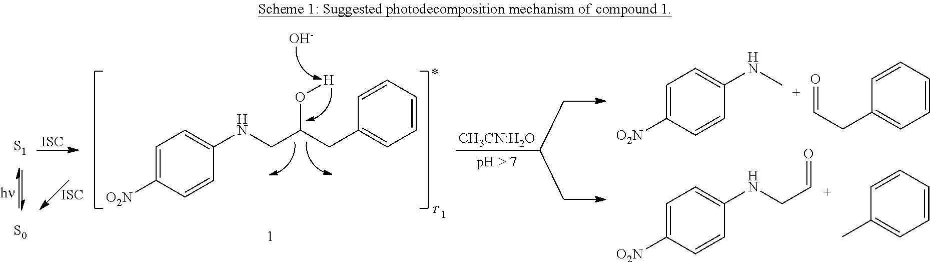

The target molecule of the method comprises of one or more substituted aromatic group(s), said substitution being selected among para, meta and ortho positions, advantageously para and/or meta positions. In one embodiment, at least one aryl group is present.

At least one aromatic group in a photolytically degradable compound according to the invention may be a nitroaryl group. Advantageously, in step c), m-nitro, o-nitro, or p-nitro substitutions at different aromatic groups are introduced. In a specific embodiment, p-nitro substitution(s), and no m-nitro substitution, are introduced in step c).

In its broadest sense, the present invention relates to synthesis of photolytically degradable compounds resembling other biologically active molecules, for example antimicrobials, such as antibacterials, or pesticides, which can undergo degradation by exposure to light. Thus, the invention embraces the synthesis of any other molecule which exhibits a biological function in its native state; and to which a compound capable of undergoing photolytic degradation may be synthesised following the teachings of the present invention. In this context, the term “light” may be what is commonly known as ‘daylight’, or light applied in an artificial and/or closed environment comprised of a specific wavelength, or a range of wavelengths.

In one embodiment the photolytically degradable compound has retained substantially all of the biological activity of the target molecule, such as at least 80%, at least 90% or at least 99% thereof. The target molecule may be any molecule which exhibits an antimicrobial, pesticidal, or antibacterial activity in its native condition. In one embodiment the target molecule may have one type of biological activity, e.g. antibacterial, and the compound with biological activity another type of biological activity, e.g. pesticidal, or vice versa.

As will be explained in further detail below (see Example 1), the chemical structure of the photolytically degradable compounds may be defined by retrosynthetic analysis of a biologically active target molecule. As well known to the person skilled in the art, retrosynthetic analysis involves transforming a target molecule into simpler precursor molecules/structures. In other words, retrosynthetic analysis comprises structural simplification of the biologically active target molecules.

In an advantageous embodiment, the nitro group is in p-position (4-position).

In one embodiment, the photolytically degradable compound comprises more than aromatic ring. Thus, Z in Formula I may be any aryl, such as

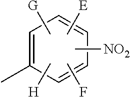

which aryl may contain a nitro group in meta or para position to the carbon attaching the Z group to the carbon chain of Formula (I), and wherein substituents E and F and G and H are defined as for A, B, C and D, respectively, while the rest of Formula I is as defined above.

Additional substitution patterns on the aromatic rings may also be envisaged within the scope of the present invention. For example, one or more of the substituents A, B, C, D, E, F, G and H may be selected from the group consisting of halogens, —CF₃, —CN, —COOH, —COOR, —CONR₂, and —R¹OR², may, enabling the making of alternative compound with biological activity. As will appear from the experimental part below, using routine methods, the skilled person is capable of synthesizing compound with biological activity as described herein, after which electron-donating and electron-withdrawing effects may be further investigated.

In one embodiment X in Formula I is F or Cl, while the rest of Formula I is as defined above.

Thus, the photolytically degradable compound according to the invention may be one or more selected from the group consisting of Compounds 1, 2, 3, 4, 5, 6, 7, 8 and 9, as described below and defined in Table 1 below.

TABLE 1
Compounds 1-9

| Compound | Structure | Target Molecule |
|---|---|---|
| 1 | 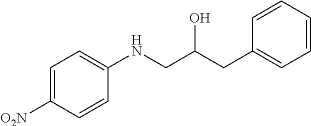 [SMILES: O=[N+]([O-])c1ccc(NCC(O)Cc2ccccc2)cc1] | |
| 2 | 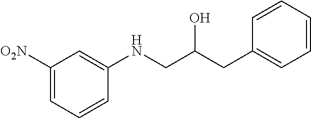 [SMILES: O=[N+]([O-])c1cccc(NCC(O)Cc2ccccc2)c1] | |
| 3 | 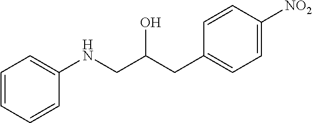 [SMILES: O=[N+]([O-])c1ccc(CC(O)CNc2ccccc2)cc1] | Flubenzuron |
| 4 | 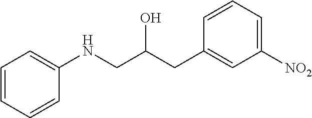 [SMILES: O=[N+]([O-])c1cccc(CC(O)CNc2ccccc2)c1] | Flubenzuron |
| 5 | | Chloramphenicol |
| 6 | 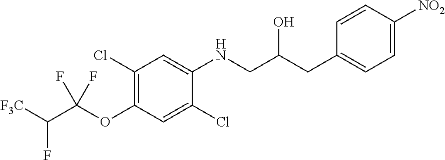 [SMILES: O=[N+]([O-])c1ccc(CC(O)CNc2cc(Cl)c(OC(F)(F)C(F)C(F)(F)F)cc2Cl)cc1] | Lufenuron |
| 7 | | Teflubenzuron |
| 8 | | Lufenuron |
| 9 | | Teflubenzuron |

Thus, as the skilled person will appreciate, the biological activity provided by the Z group of Formula I may be antimicrobial activity, antibiotic activity, pesticidal activity or the like. Such groups or entities may be known as such, but have not until now been included in a compound specifically designed to provide photolytically degradable properties.

A specific example of a photolytically degradable compound according to the invention, wherein the Z is a beta lactam, is defined by Formula II below:

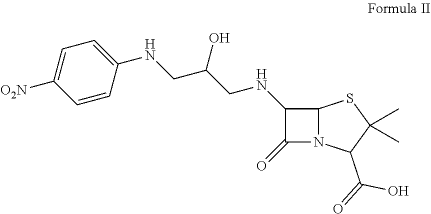 [SMILES: CC1(C)SC2C(NCC(O)CNc3ccc([N+](=O)[O-])cc3)C(=O)N2C1C(=O)O]

As the skilled person will appreciate, since the advantages of the present invention are closely related to the chemistry, there may be any organic molecule that fulfils the structural criteria presented herein, such a pesticide, exemplified by diflubenzuron or teflubenzuron; or an antibiotic molecule, exemplified by chloramphenicol.

The reaction used in the present invention to degrade the active molecule by light is of the photo-retro-aldol type proceeding through a heterolytic cleavage of an aliphatic C—C bond. Wan and Muralidharan (Wan, P.; Muralidharan, S. J. Am. Chem. Soc. 1988, 110 (13), 4336-4345) observed this transformation with p- and m-nitro-substituted phenethyl alcohols at different pH conditions and varying substituents in 1-position (R group), as appears from Scheme 2 below. In one embodiment a compound with biological activity synthesized according to the invention is capable of heterolytic cleavage as a result of exposure to light.

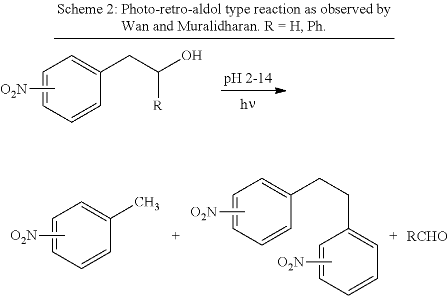

With the 1-phenyl substituted phenethyl alcohols, the reactions were more or less pH independent as conversions of 30-60% were achieved at pH 2-14. However, with a proton instead of a phenyl group, pH above 11 was required. The mechanism, see Scheme 3, suggests that the reactive triplet state undergoes a retro-aldol reaction through abstraction of a proton, by either water or hydroxide, forming p-nitrobenzyl carbanion and formaldehyde (Wan, P.; Muralidharan, S. J. Am. Chem. Soc. 1988, 110 (13), 4336-4345).

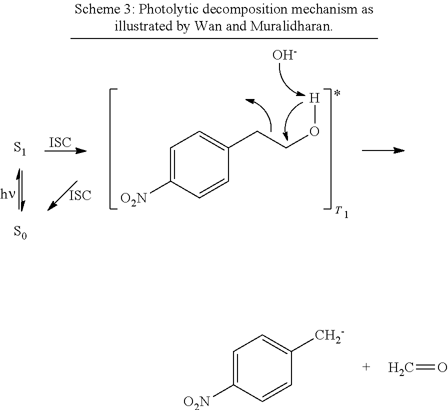

With this in mind, the present inventors decided to apply this chemistry to make modified flubenzurons. By simplifying the structure, it was possible to construct a series of non-complex compounds that contain similar structural elements, which would be easy to synthesize. The compounds presented below, in Scheme 4, have the two aromatic groups and a chain of equal length and were therefore considered to be suitable test compounds for a proof-of-concept study for the photodegradation.

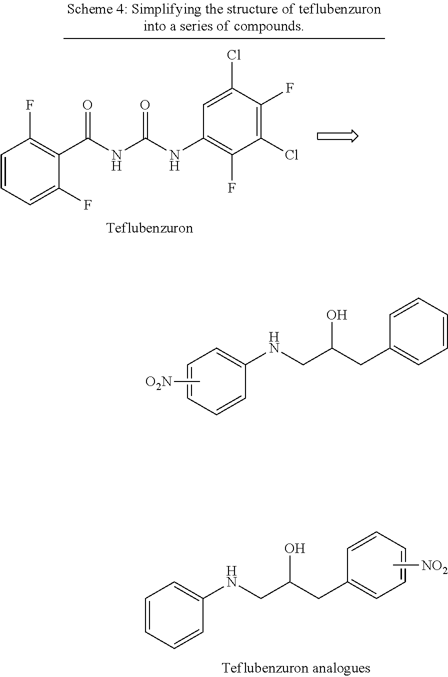

In order to investigate isolated effects from the electron-withdrawing capability of the nitro group, the starting point was with mono-substituted systems. A simple beginning was therefore to include p- and m-nitro substitution separately on the two phenyl rings, which were easily made from commercially available and cheap starting materials, such as nitroaniline and iodonitrobenzene, see Scheme 5. Once the four compounds are obtained, their photochemical properties and decomposition patterns were investigated.

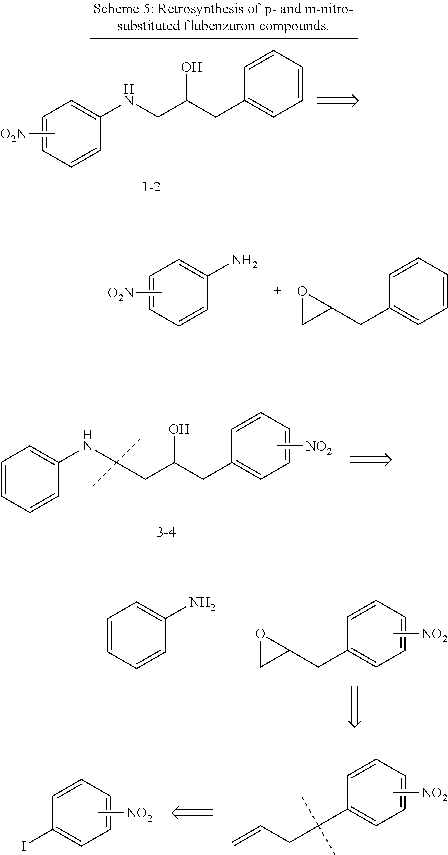

Compounds 6-9 with antibacterial properties could be prepared by a Suzuki-Miyaura cross-coupling reaction with allyl boronic acid pinacol ester, using a method described by Kotha et al (Kotha, S.; Behera, M.; Shah, V., Synlett 2005, 12, 1877-1880). Subsequent treatment with mCPBA gave the corresponding epoxide. A Lewis acid-promoted epoxide ring-opening reaction using 5 M lithium perchlorate-diethyl ether (LPDE) solution gave compound 6. Compounds 7-9 were prepared by a selective meta nitration followed by a Stille cross-coupling with allyltributylstannane and subsequent epoxidation with mCPBA. Lewis acid-promoted epoxide ring opening with aniline A₁-A₃ gave aminols 7-9, see Scheme 6.

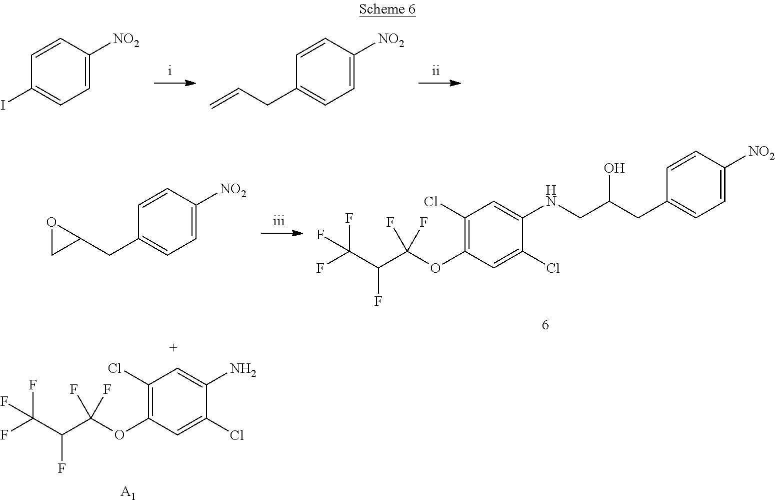 [SMILES: O=[N+]([O-])c1ccc(I)cc1 ; C=CCc1ccc([N+](=O)[O-])cc1 ; O=[N+]([O-])c1ccc(CC2CO2)cc1 ; O=[N+]([O-])c1ccc(CC(O)CNc2cc(Cl)c(OC(F)(F)C(F)C(F)(F)F)cc2Cl)cc1 ; Nc1cc(Cl)c(OC(F)(F)C(F)C(F)(F)F)cc1Cl ; "A1"]

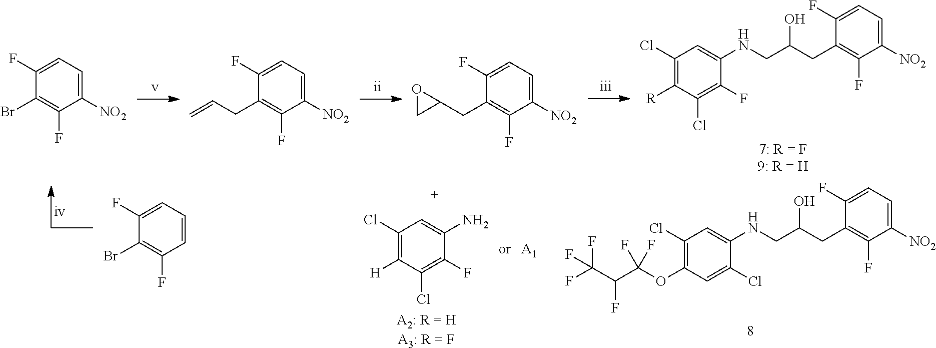

Complete synthesis of compounds 6-9. Reagents and conditions: (i) Pd(PPh₃)₄, CsF, AllylBpin, THF, reflux; (ii) mCPBA, DCM, rt; (iii) 5 M LPDE, 40° C.; (iv) H₂SO₄, HNO₃, 0° C.; (v) Pd(PPh₃)₄, Bu₃SnAllyl, DMF, 110° C.

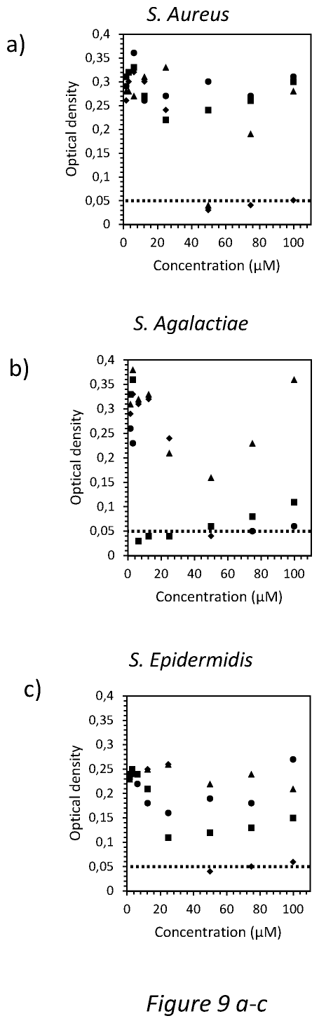

Compounds 6-9 were analysed for antibacterial activity, see FIG. 9. All compounds 6-9 showed antibacterial activity, and can be photolytically degraded, as discussed further in the experimental section. The degraded parent compounds [Compounds 6-9] showed no antimicrobial activity. Hence, the compounds with biological activity described herein are antibacterial and can be used for antibacterial applications. One advantage with the invention is that the active compounds are photolytically degradable another advantage is that the decomposition products are inactive at 100 μM concentration, see Table 2 and further details in the experimental section.

The idea concerning photolytic decomposition has been extended to antibiotics, as waste water treatment plants do not have the necessary design to completely remove these substances. For example, ciprofloxacin has been detected in the effluent from a treatment plant, and another study investigated commonly used antibiotics in the U.S. and showed that even though their concentration decreased during wastewater treatment, they persist. Another antibacterial molecule that causes similar issues is chloramphenicol, a broad-spectrum antibiotic. A closer look at the structure reveals some similarities to the compounds resembling flubenzuron discussed above. This would make it possible to construct a compound with similar functionalities (compound 5), see Scheme 7.

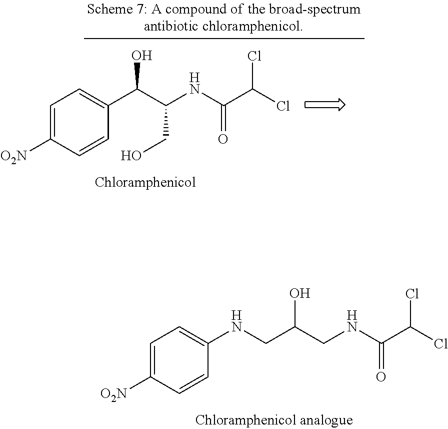 [SMILES: O=C(N[C@H](CO)[C@H](O)c1ccc([N+](=O)[O-])cc1)C(Cl)Cl ; O=C(NCC(O)CNc1ccc([N+](=O)[O-])cc1)C(Cl)Cl]

Once again, the target molecule was easily made from readily available and cheap starting materials, as described in further detail below under ‘Synthesis’. Through a few intuitive disconnections and a functional group interconversion (FGI), a simple retrosynthetic analysis illustrates that Compound 5 could be synthesized through three steps, see scheme 8.

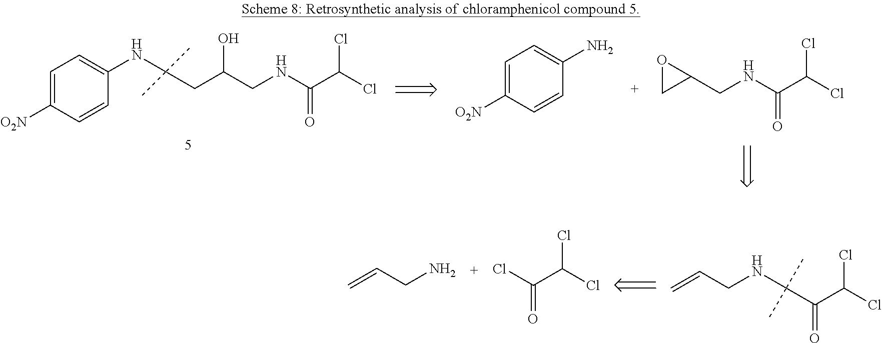

Thus, another aspect of the invention is a method of reducing the load of antimicrobial molecules in waste or drinking water, wherein water including an antibacterial molecule obtained and synthesized according to the method of the invention is subjected to light to photolytically degrade any remaining pharmaceutically active molecule. The pH of the wastewater may be adjusted as appropriate, for example to above 7, or to about 11.

All details regarding the reactions and chemistry discussed herein in relation to the first aspect are equally applicable to this second aspect and other aspects.

A further aspect of the invention is a method of controlling salmon lice in aquaculture, wherein a pesticide compound obtained and synthesized according to the method of the invention is used to treat salmon in an aqueous culture to reduce or eliminate the occurrence of salmon lice, after which treatment the water originating from said culture is subjected to light to photolytically degrade any remaining pesticide. The pH of the wastewater may be adjusted as appropriate, for example to above about 7. The skilled person will appreciate that different pH values may be used, depending e.g. on the chemistry of the parasiticide or pesticide.

In a specific embodiment, the present invention relates to a method of selectively synthesizing a photolytically degradable compound with biological activity, which method comprises the steps of
a) identifying a biologically active target molecule comprising one or more substituted aromatic group(s);
b) selecting a suitable starting material for synthesis of compound resembling said target molecule, which starting material presents at least the aromatic group(s) of the target molecule; and
c) synthesizing a compound by introducing m-nitro or p-nitro substitution(s) in at least one aromatic group of the starting material selected in step b), wherein the compound comprises at least one C—N, C—S or C—O bond and optionally at least one benzylic C—C bond capable of heterolytic cleavage as a result of exposure to light at basic pH in aqueous environment.

The biologically active target molecule may be a molecule characterized by its capability to destroy and/or harm another compound, such as a microbe or a parasite, such as a molecule selected from the group consisting of antimicrobial molecules and pesticides.

In this specific embodiment of the invention, the compound with biological activity is defined by the general Formula I:

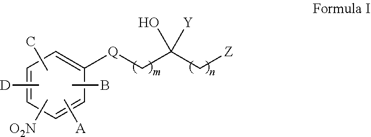

where in the aryl group attached to the Q, the nitro group is meta or para to the carbon atom attached to Q;
and wherein
at least one of A and B and C and D is
   O(CH₂)ₒ(CX₂)ₚ(CH₂)_{q}(CHX)ᵣ(CH₂)ₛ(CX₂)ₜ(CH₂)ᵤ(CX₃)ᵥ(CHX₂)_{w}(CH₂X)ₓ(CH₃)_{y} where
   o=0-5, p=0-5, q=0-5, r=0-5, s=0-4, t=0-4, u=0-4, v=0-1, w=0-1, x=0-1, y=0-1, provided that in each compound, only one of v, w, x, and y are 1 while the other three are 0, and
   where X is F and/or Cl,

while the other three of A, B, C and D are
1) each independently selected from the group consisting of H, F, Cl, CF₃, C₁-C₆ alkyl, alkoxy with a C₁-C₆ alkyl, NH₂, NHR¹, NR¹R², and [NR¹R²R³]⁺ where R¹, R² and R³ are each selected from a group consisting of H and branched or unbranched C₁-C₆ alkyl, and
2) can be randomly attached to the four remaining carbon atoms in the phenyl ring available for substitution;
where n=0-4
Q is either
   NH, NR⁴, [NR⁴R⁵]⁺, NOH, NOR⁴, NNH₂, NNHR⁴, [NNHR⁴R⁵]+, NNR⁴R⁵, and [NNR⁴R⁵R⁶]⁺ where R⁴, R⁵, and R⁶ are a branched or unbranched C₁-C₃ alkyl;
   or O;
   or S, [SR⁴]⁺, SO and SO₂;
Y is selected from a group consisting of H and branched or unbranched C₁-C₆ alkyl;
Z is either
   H, or
   NHC(O)R⁷
      wherein R⁷ is either R⁵, or monohalogenated-perhalogenated derivatives of R⁵,
         wherein the halogen is F and/or Cl;
aryl; or

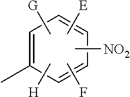

which aryl may contain a nitro group in meta or para position to the carbon attaching the Z group to the linear carbon chain of Formula (I).
and wherein
substituents E and F and G and H are defined as for A, B, C and D, respectively.

Specifically, Z in Formula I may either be aryl; or

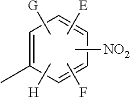

which aryl may contain a nitro group in meta or para position to the carbon attaching the Z group to the carbon chain of Formula (I).
and wherein
substituents E and F and G and H are defined as for A, B, C and D.

X in Formula I above may be Cl.

All details and applications discussed elsewhere in the present description will also apply to this last-mentioned specific embodiment of the invention.

### DETAILED DESCRIPTION OF THE DRAWINGS

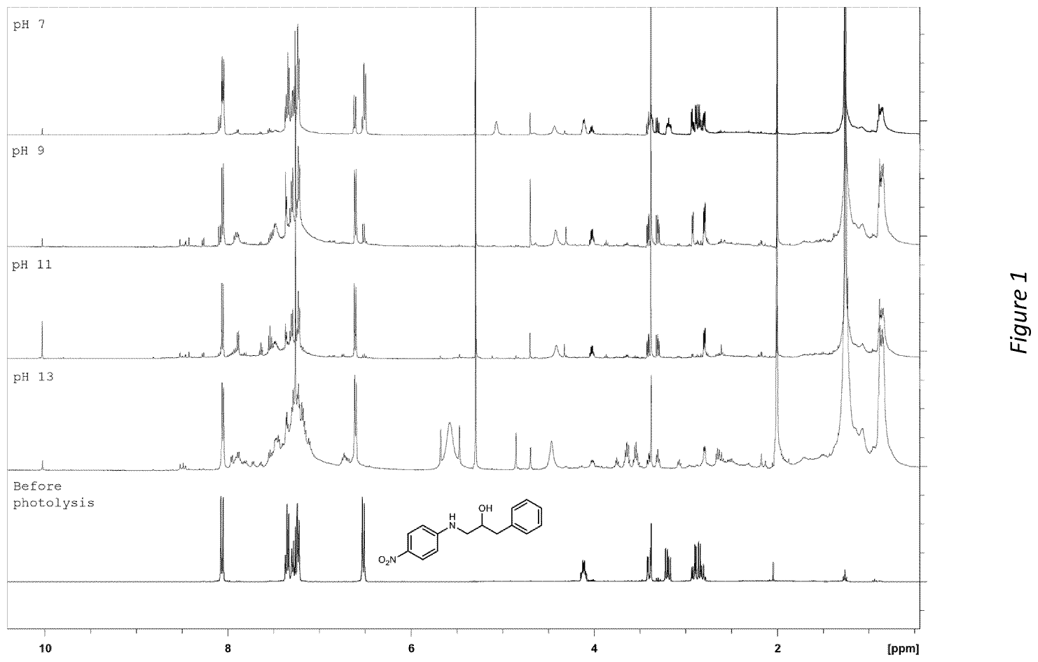

FIGS. 1-9 all illustrate results obtained as described in the experimental section below:

FIG. 1: shows the ¹H-NMR spectra before and after photolysis of compound 1 (see Table 1 above). The y-axis shows relative intensity and the X-axis shows [ppm]. From top to bottom, the spectra are obtained at pH 7; pH 9; pH 11; pH 13; and before photolysis, respectively. 100% conversion was obtained at pH 11; and 13. 50% conversion was obtained at pH 7.

With 32% conversion at pH 7, after 2 hours irradiation indicates that this would be fully decomposed in the course of ca. 6 hours.

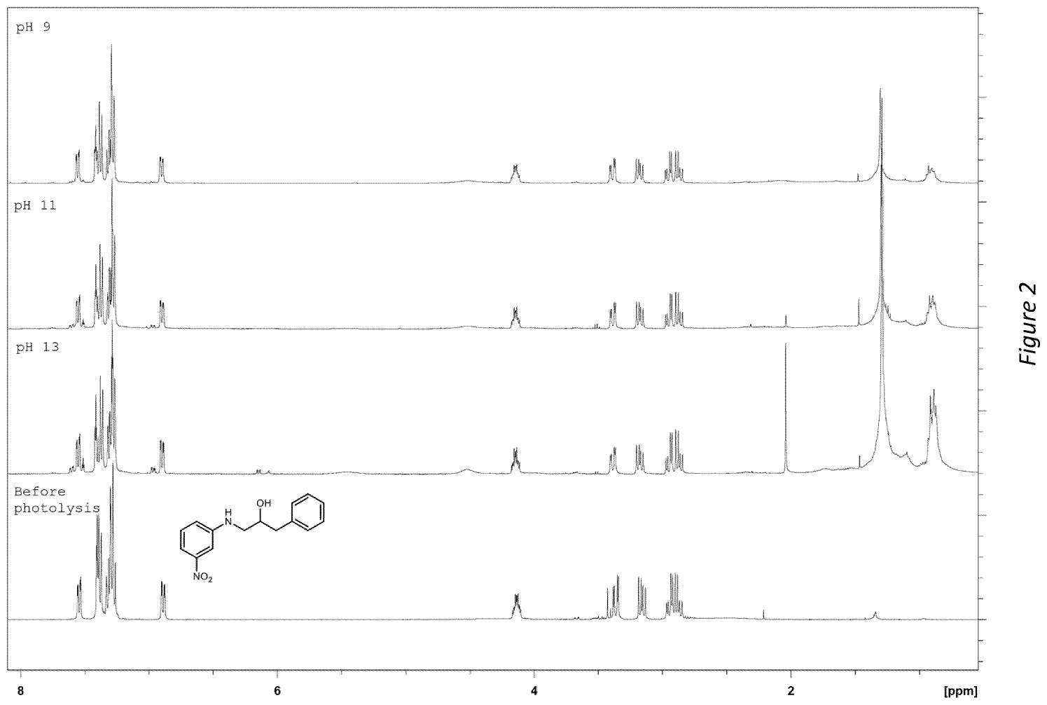

FIG. 2: ¹H-NMR spectra before and after photolysis of compound 2 (see Table 1 above). The y-axis shows relative intensity and the X-axis shows [ppm]. From top to bottom, the spectra are obtained at pH 9; pH 11; pH 13; and before photolysis, respectively. 17% conversion was obtained at pH 13. This result highlights the advantages of having the nitro group in p-position (4-position).

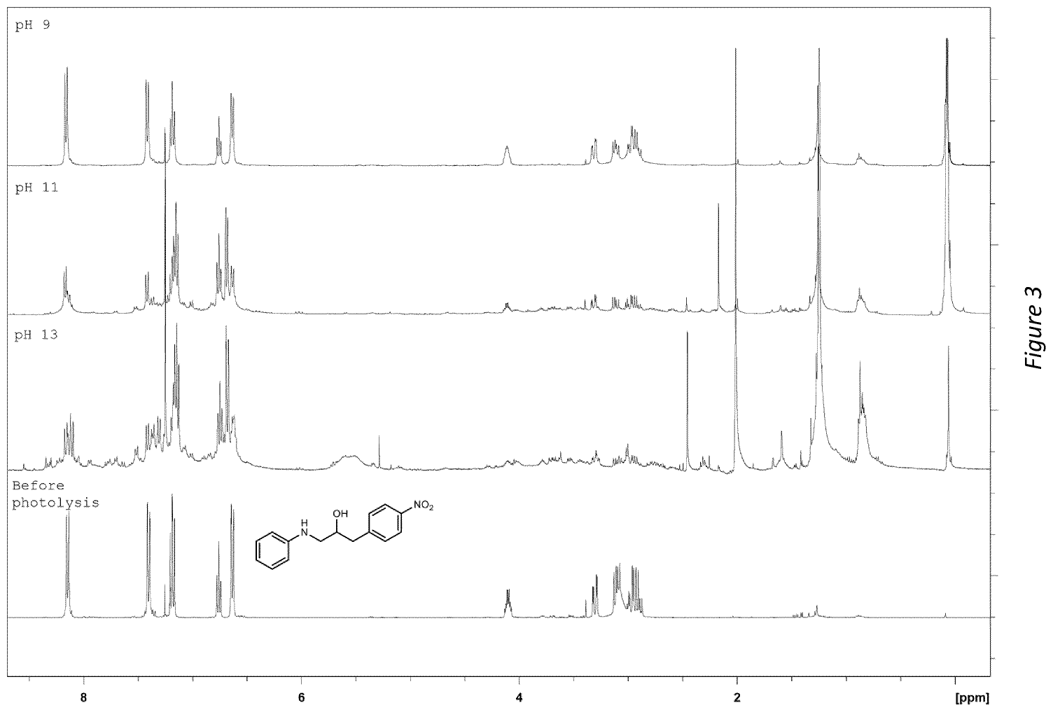

FIG. 3: ¹H-NMR spectra before and after photolysis of compound 3 (see Table 1 above). From top to bottom, the spectra are obtained at pH 9; pH 11; pH 13; and before photolysis, respectively. The y-axis shows relative intensity and the X-axis shows [ppm]. 40% conversion was obtained at pH 13.

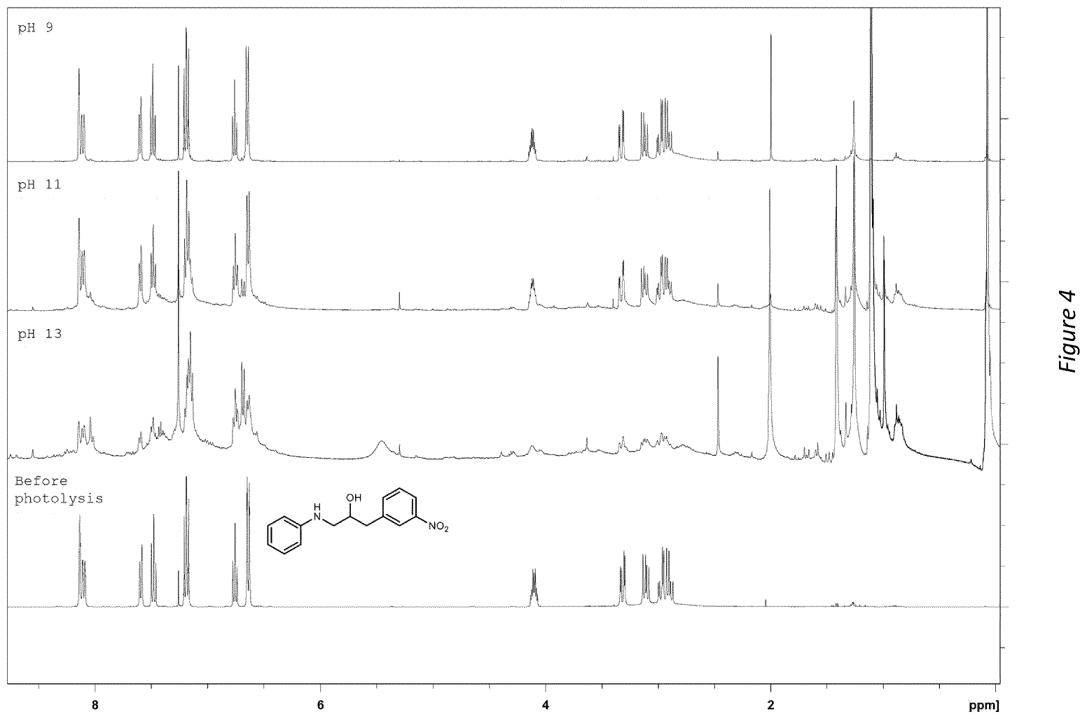

FIG. 4: ¹H-NMR spectra before and after photolysis of compound 4 (see Table 1 above) compared with p-nitroaniline. From top to bottom, the spectra are obtained at pH 9; pH 11; pH 13; and before photolysis, respectively. The y-axis shows relative intensity and the X-axis shows [ppm]. 56% conversion was obtained at pH 13.

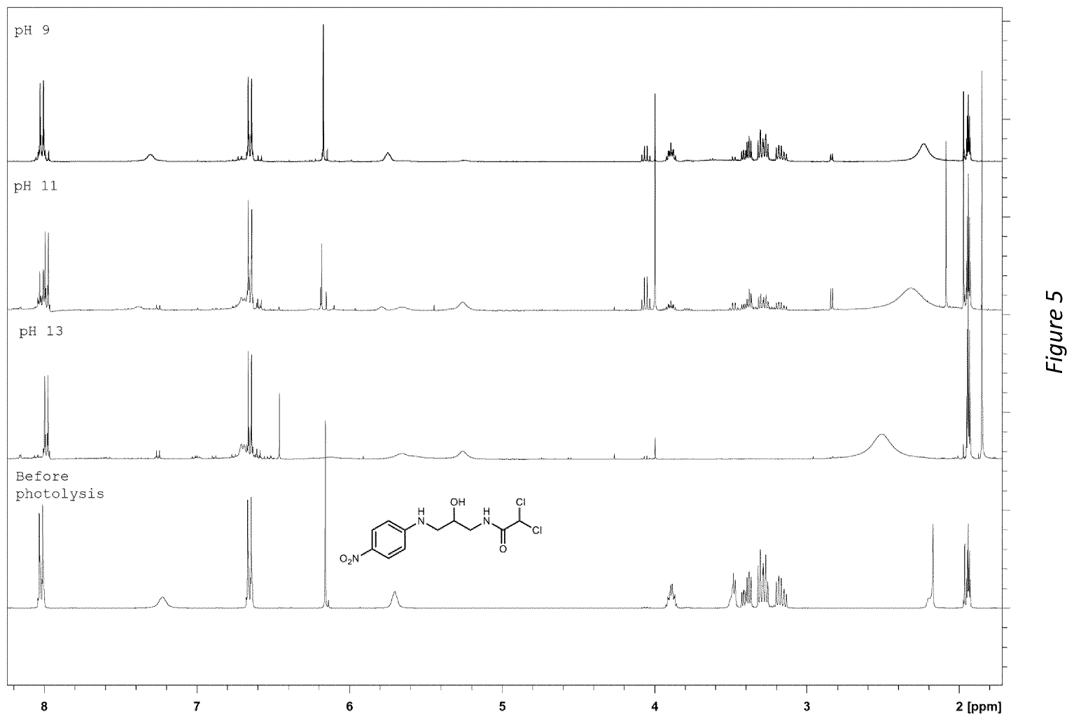

FIG. 5: ¹H-NMR spectra before and after photolysis of compound 5 (see Table 1 above). From top to bottom, the spectra are obtained at pH 9; pH 11; pH 13; and before photolysis, respectively. The y-axis shows relative intensity and the X-axis shows [ppm]. 100% conversion was obtained at pH 13, and 68% conversion was obtained at pH 11.

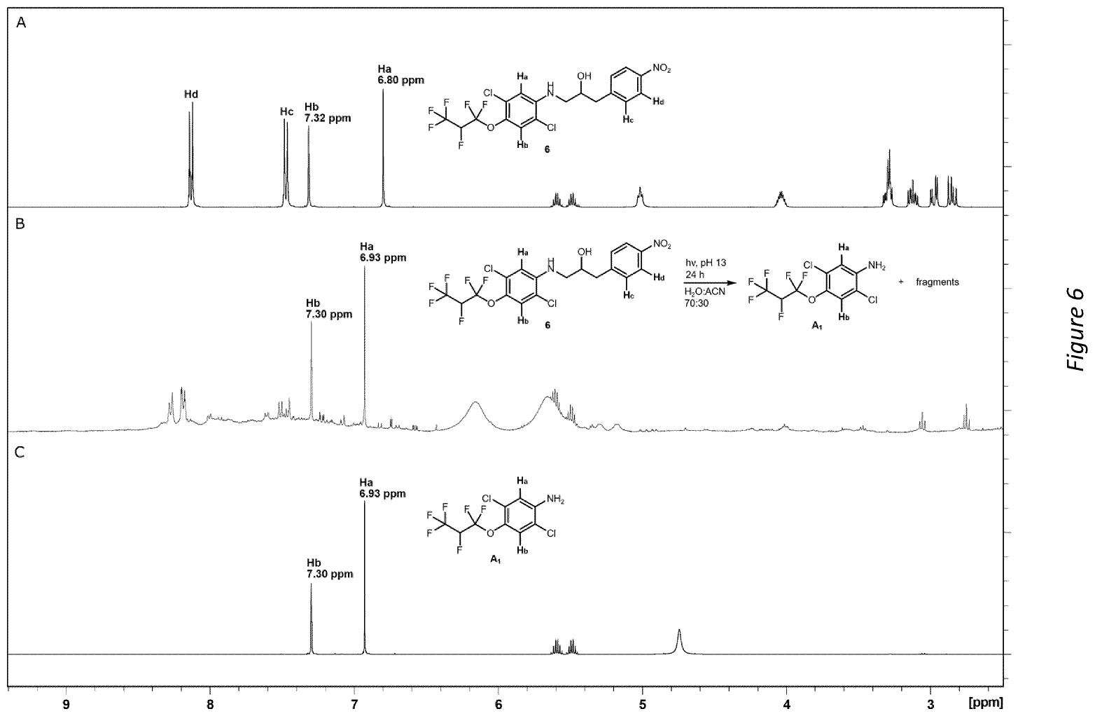

FIG. 6: ¹H-NMR spectra of (A) compound 6 before irradiation, (B) decomposition product, and (C) aniline A1. The y-axis shows relative intensity and the X-axis shows [ppm].

FIG. 6 highlights the formation of aniline A1 as one of the decomposition products.

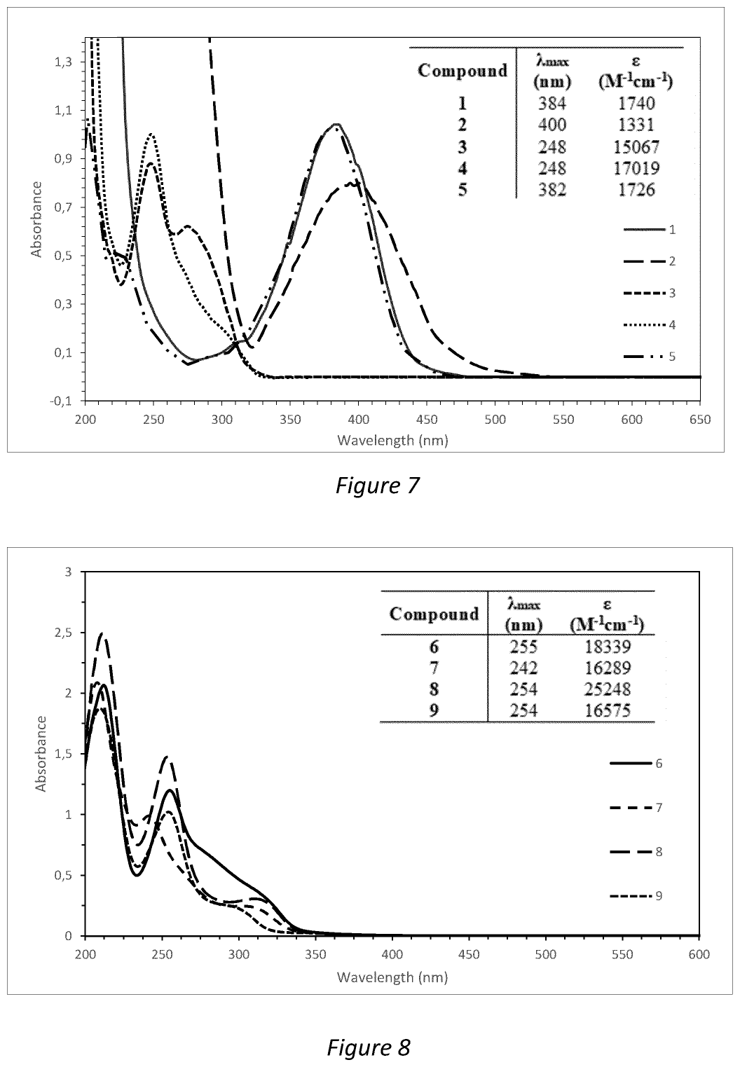

FIG. 7: UV-vis spectra of compounds 1-5. The UV-vis data for compound 2 (Table 1) shows a λₘₐₓ of 400 nm, e.g. a bathochromic shift of 16 nm. The y-axis shows absorbance in a.u. and the X-axis shows wavelength in nm.

It appears that compound 2 is less exposed to irradiation with a medium pressure mercury lamp, which emits mainly around 312, 366 and 575 nm. See photolysis section for details discussion.

FIG. 8: UV-vis spectra of compounds 6-9 at 0.06 mM with corresponding λₘₐₓ and ε, determined as the slope of standard curves from concentrations 0.02, 0.04, 0.06, and 0.08 mM for each compound (R²≥0.999). The y-axis shows absorbance in a.u. and the X-axis shows wavelength in nm.

FIG. 8 highlights that compounds 6-9 all have λₘₐₓ at ca 250 nm.

FIG. 9 a-c. Minimum inhibitory concentration (MIC) plots for Compound 6 (•), Compound 7 (▴), Compound 8 (▪), and Compound 9 (♦) against S. Aureus (FIG. 9a), S. Agalactiae (FIG. 9b), and S. Epidermis (FIG. 9c). Black stapled lines at 0.05 for MIC indicate activity threshold values. The y-axis shows optical density and the X-axis shows concentration in μM.

### EXPERIMENTAL

The present examples are provided for illustrative purposes only, and are not to be construed as limiting the scope of the present invention as defined by the appended claims. All references provided below and elsewhere in the present application are hereby included herein via reference.

### Compounds 1-5

### Synthesis

Using p- and m-nitroaniline as nucleophiles can be problematic and sluggish due to the poor nucleophilicity owing to the electron-withdrawing effect from the nitro group. This was indeed observed when the reaction was attempted according to a microwave-assisted aminolysis reported by Lindsay et al (Lindsay, H .; Desai, H .; D'Souza, B .; Foether, D.; Johnson, B. Synthesis 2007, 6, 902-910). However, these reactions are significantly speeded up if they are performed in 5 M lithium perchlorate-diethyl ether solution as reported by Heydari et al. (Heydari, A.; Mehrdad, M.; Maleki, A.; Ahmadi, N. Synthesis 2004, 10, 1563-1565). High concentration of the weakly Lewis acidic oxophilic Lit ion is sufficient in order to facilitate the reaction. Additionally the reaction is performed at room temperature and yields the desired products (compounds 1 and 2) in good yields (Scheme 9). A side product resulting from a secondary aminolysis was detected in trace amounts.

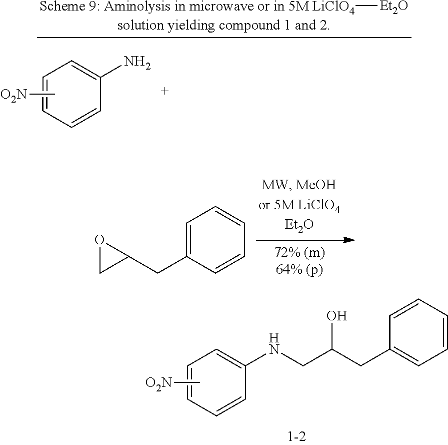

For compounds 3 and 4 a slightly longer synthetic pathway was required as the nitro substituted epoxides were not commercially available. A simple Suzuki-Miyaura palladium-catalyzed cross-coupling reaction with allyl boronic acid pinacol ester and an aryl iodide, followed by standard epoxidation with mCPBA, and subsequent epoxide aminolysis with aniline yielded the desired products in 20-30% yield over three steps (Scheme 10).

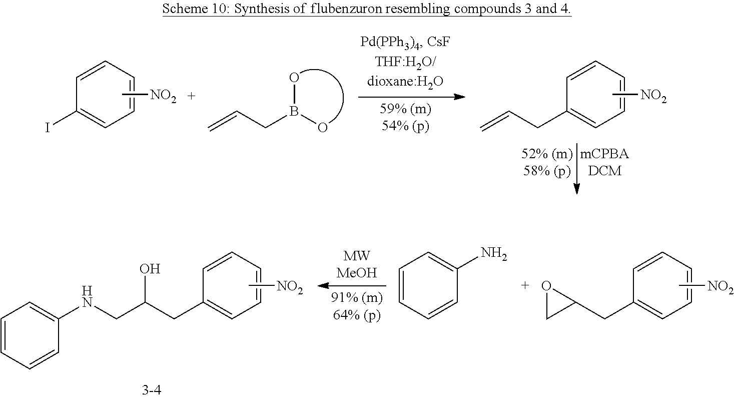

Acylation with dichloroacetyl chloride under standard conditions with triethylamine in dichloromethane yielded N-allyl-2,2-dichloroacetamide in 99% yield (Scheme 11). Exposing the alkene to mCPBA in DCM resulted in the epoxide which was ring opened with p-nitroaniline in 5 M lithium perchlorate-diethyl ether solution in an overall yield of 62% over three steps.

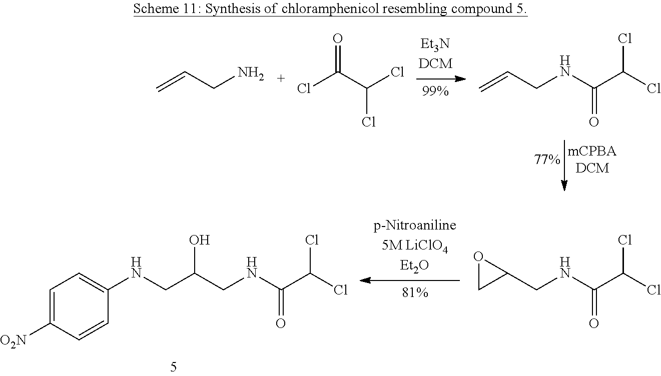

### Photolysis

With aminol 1-5 at hand, the spectroscopic properties, such as λₘₐₓ and ε, were determined and are summarized in Table 2 and FIG. 7. As illustrated in the UV-vis spectra, where the nitro group is positioned greatly influences the λₘₐₓ. With nitro substitution on the aniline these values are around 380 and 400 nm, whereas for the two other compounds a hypsochromic shift is observed all the way down to 248 nm.

TABLE 2
Spectroscopic data for compounds 1-5.

| | | λₘₐₓ | E |
|---|---|---|---|
| Compound | Structure | (nm) | (M⁻¹cm⁻¹) |
| 1 | | 384 | 1740.0 |
| 2 | | 400 | 1331.3 |
| 3 | | 248 | 15066.6 |
| 4 | | 248 | 17018.7 |
| 5 | | 382 | 17263.9 |

Following the procedure by Wan and Muralidharan (Wan, P.; Muralidharan, S. J. Am. Chem. Soc. 1988, 110 (13), 4336-4345) aminol 1 (compound 1) was photolysed in acetonitrile and water (7:3, v:v) at the appropriate pH using a 125 W medium pressure mercury vapour lamp. Seven experiments were performed from pH 13 to pH 1 with a concentration of around 0.68 mM, and each reaction was irradiated for. 2 hours Following liquid-liquid extraction with DCM, all reaction mixtures were analysed with ¹H-NMR spectroscopy. Initially it was expected that a mechanism similar to the nitrophenethyl alcohols (Scheme 3) would occur and the expected products would therefore be N-methyl-p-nitroaniline, phenylacetaldehyde, 2-(p-nitroaniline)acetaldehyde, and toluene

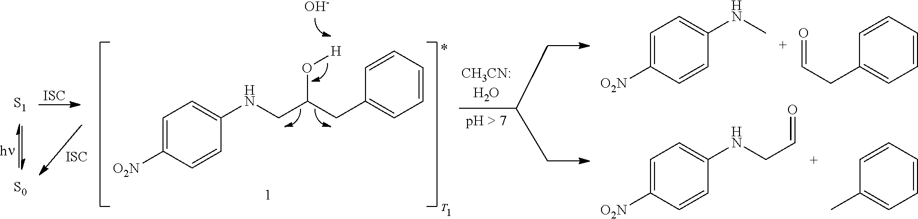

(Scheme 12).

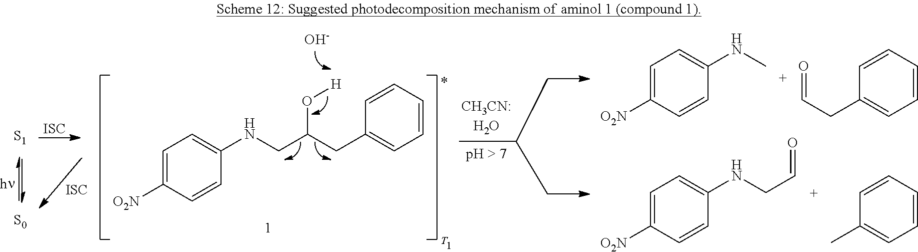

However, none of these compounds were detected, but as seen in the ¹H-NMR spectra, a 100% conversion was observed at pH 13 and 11 after 2 hours. As the pH decreases, so does the conversion and it stops completely at pH 5. Reference experiments were also run with the same conditions in the dark, and showed no conversion. Because of this, it seems likely that following excitation to the singlet state and intersystem crossing (ISC) to the triplet state, a proton is abstracted and initiates decomposition. Two products were identified from the NMR data; p-nitroaniline and benzaldehyde. This means that there are two carbon atoms in the starting material unaccounted for (Scheme 13). A reasonable hypothesis is that they remain in the aqueous phase during extraction as formaldehyde or glyoxal.

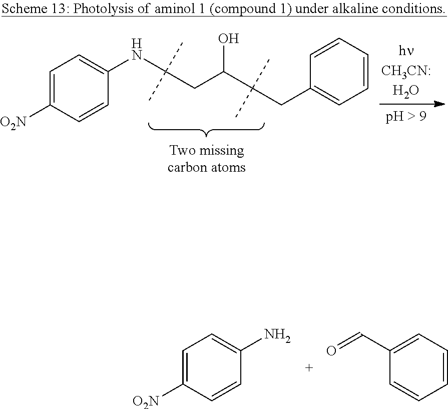

With the nitro group in m-position the conversion was not as good. At pH 13 and 11 a conversion of 17% was observed and the compound remained stable at pH values lower than 11. The UV-vis data for compound 2 (Table 2 and FIG. 7) shows a λₘₐₓ of 400 nm, e.g a bathochromic shift of 16 nm. This means that the compound is less exposed to irradiation with a medium pressure mercury lamp, which emits mainly around 312, 366 and 575 nm (Ushio America Inc. (2019) UVH Medium Pressure Mercury Arc).

Additionally, the nitro group in p-position will have a significant mesomeric effect, compared to having it in m-position, easily illustrated by the resonance structure (Scheme 14). Therefore, it might not sufficiently influence the pKₐ value of the hydroxyl proton, and no reaction occurs.

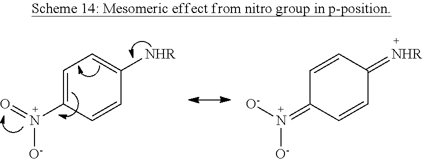

With the nitro group on the other phenyl ring, no photolytic decomposition was observed, regardless of the pH (Scheme 15). This is most likely because medium-pressure mercury lamps do not irradiate at 248 nm, which is the λₘₐₓ value for these compounds, Table 1). However, using a different lamp, such as a low-pressure mercury lamp, a reaction will take place, since they have a strong emission at 254 nm.

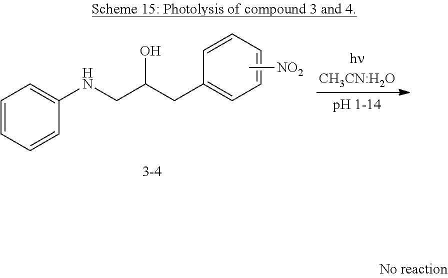

The compound resembling chloramphenicol reacted similarly as compound 1, and p-nitroaniline was indeed detected. Full conversion was achieved at pH 13 and 60% at pH 11. At lower pH values, the compound remained stable. However, no aldehyde signal was observed and the ¹H-NMR data in the aliphatic region of the spectrum remain inconclusive at this point.

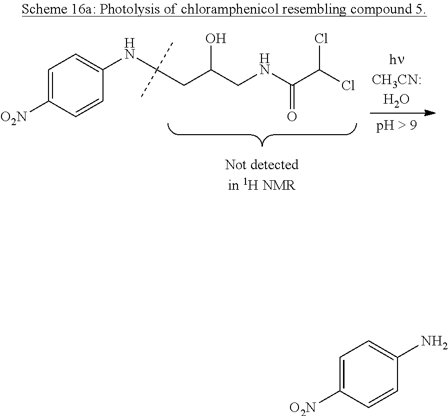

Compounds 1-5 were tested against five different bacteria (E. faecalis, E. coli, P. aeruginosa, S. aureus, and S. agalactiae), but displayed no antimicrobial activity. In order to make a compound that more closely resembles the structure of chloramphenicol that also includes the same ethanol-substituted aniline as compounds 1-5, a hydroxylamine seems to be a potential solution (Scheme 16b).

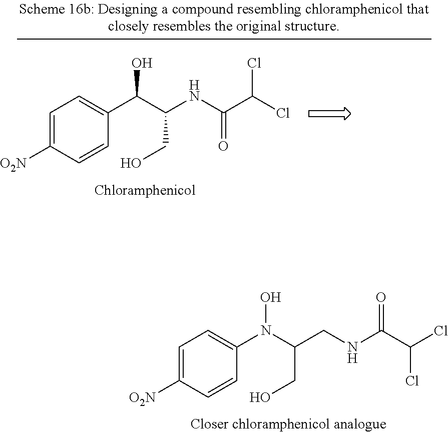 [SMILES: O=C(N[C@H](CO)[C@H](O)c1ccc([N+](=O)[O-])cc1)C(Cl)Cl ; O=C(NCC(CO)N(O)c1ccc([N+](=O)[O-])cc1)C(Cl)Cl]

A retrosynthetic approach to a compound resembling chloramphenicol, compound 10, can start with a disconnection on the aniline (Scheme 17). Hydroxylamine is a good nucleophile and is commercially available, and it is therefore natural to make another disconnection there. Functional group interconversion to an alcohol results in a vicinal diol, which is commonly synthesised from the corresponding alkene.

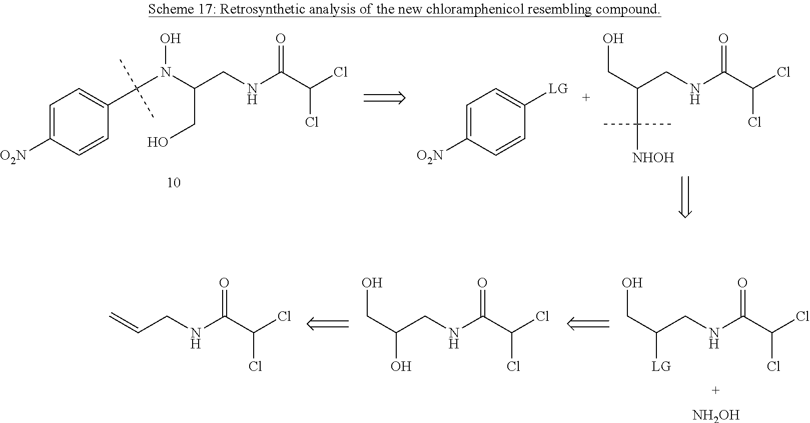

Dihydroxylation with potassium osmate and K₃Fe(CN)₆ as co-oxidant gave the vicinal diol in excellent yield (92%). In order to selectively tosylate the secondary alcohol, a benzoylation was required initially as the benzoyl group usually reacts selectively on primary alcohols, whereas tosylates do not. However, dibenzoylation occurred, explaining the low yield (39%). The tosyl group was then installed and a nucleophilic substitution reaction with O-trimethylsilyl hydroxylamine is the next step to be performed (Scheme 18).

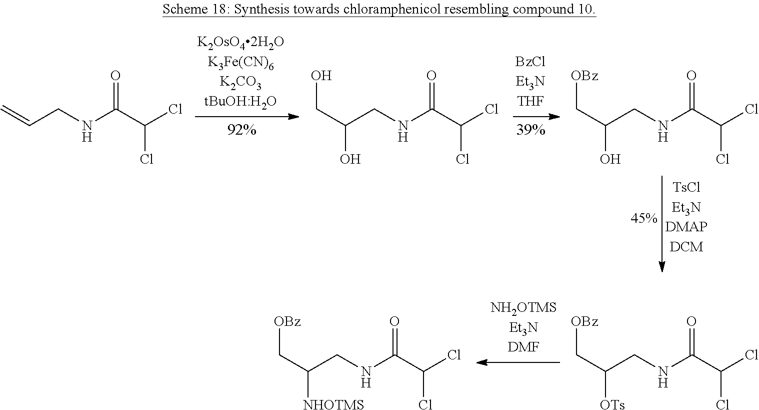

It would also be possible to exchange the hydroxylamine moiety for a hydrazine (Scheme 19). The synthetic pathway would be similar for compound 10, except that hydrazine would be used as nucleophile in the fourth step instead of hydroxylamine.

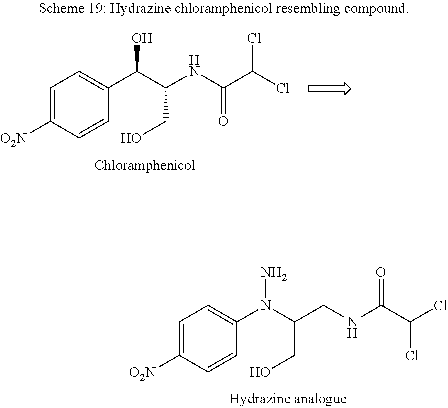 [SMILES: O=C(N[C@H](CO)[C@H](O)c1ccc([N+](=O)[O-])cc1)C(Cl)Cl ; NN(c1ccc([N+](=O)[O-])cc1)C(CO)CNC(=O)C(Cl)Cl]

Two additional compounds, though structurally more distant than the hydroxylamine-and the hydrazine analogue, should easily be made from O-TMS-N-nitrophenylhydroxylamine or N-nitrophenyl-Bochydrazine with the epoxide prepared according to (Scheme 20).

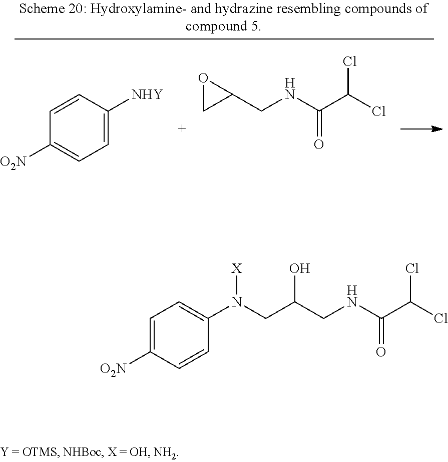

### Compounds 6-9

### Synthesis

Compounds 6-9 were prepared. To this end a Suzuki-Miyaura cross-coupling reaction with allyl boronic acid pinacol ester, using a method described by Kotha and co-workers (Kotha, S.; Behera, M.; Shah, V., A. Synlett 2005, 12, 1877-1880). Subsequent treatment with mCPBA gave the corresponding epoxide. A Lewis acid-promoted epoxide ring-opening reaction using 5 M lithium perchlorate-diethyl ether

(LPDE) solution gave aminol compound 6. The remaining three compounds were prepared by a selective meta nitration followed by a Stille cross-coupling with allyltributylstannane and subsequent epoxidation with mCPBA yielding the required epoxide E. Lewis acid-promoted epoxide ring opening with anilines A-C gave aminols 7-9 (Scheme 21).

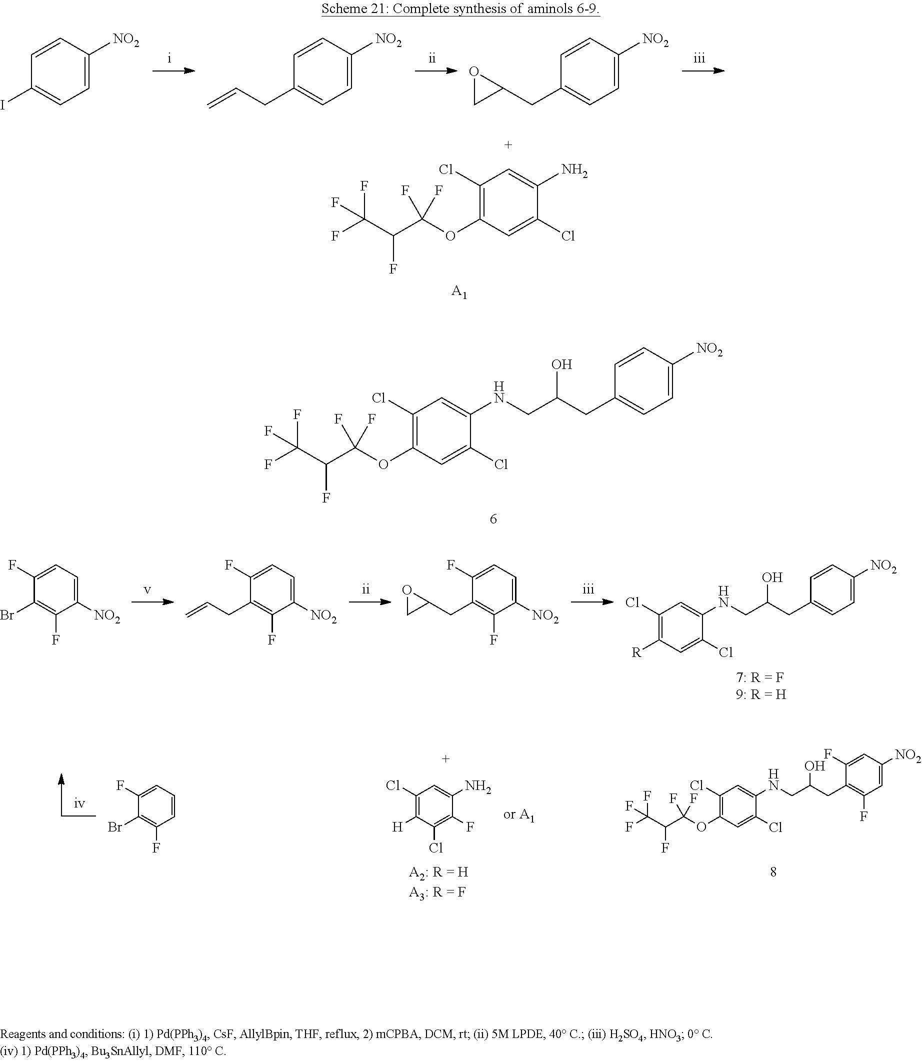

### Photolysis

Aminol 6-9 (compounds 6-9) were dissolved in acetonitrile and diluted with a sodium hydroxide solution at pH 13 (30/70, V/V). Since all the compounds have strong absorption bands around 250 nm (FIG. 8), a 6 W low-pressure mercury vapor lamp was chosen as the irradiation source, since it emits mainly at 254 nm. All reactions were carried out in a 75 mL gas inlet flask (˜0.7 mM) under a constant stream of nitrogen with a quartz well cold finger for 24 hours.

FIG. 8 shows the UV-vis spectra of compound 6, compound 7, compound 8 and compound 9 at 0.06 mM with corresponding λₘₐₓ and ε, determined as the slope of standard curves from concentrations 0.02, 0.04, 0.06, and 0.08 mM for each compound (R²≥0.999).

Following aqueous workup, the reaction mixtures were analysed with ¹H-NMR spectroscopy. For compound 6, complete conversion was observed as the two singlets at 6.80 and 7.32 ppm (Hₐand H_{b}), has shifted significantly to 6.93 and 7.30 ppm, which corresponds with 2,5-dichloro-4-(1,1,2,3,3,3-hexafluoropropoxy)aniline. The photodegradation reaction for compound 6 can be seen in Scheme 21. Additionally, the two doublets at 7.47 and 8.13 ppm (H_{c} and Hₐ) have disappeared into a complicated multiplet, and the aliphatic protons around 3 ppm are no longer visible (FIG. 6). FIG. 6 shows ¹H-NMR spectra of (A) compound 6 before irradiation, (B) decomposition product, and (C) aniline A1. A similar result was observed for compound 7 and compound 9, but for compound compound 8, a complete degradation was not achieved under these conditions. The ratio of signal Hₐ for the starting material and the degradation product revealed a 56% conversion.

### Antibacterial Activity

With compounds 6-9 and the crude degradation mixtures in hand, the minimum inhibitory concentration (MIC) was screened against a number of gram-positive and gram-negative bacteria, including E. faecalis, E. coli, P. aeruginosa, S. aureus, S. agalactiae, and S. epidermidis. To determine the activity, optical density (OD) at 600 nm was measured to estimate the concentration of bacteria at concentrations from 1.6 -100 μM. Values below 0.05 are counted as active and indicates inhibition of growth, illustrated by plotting OD against the concentration for compounds 6-9, (FIG. 9). FIG. 9 a-c shows minimum inhibitory concentration plots for compound 6 (•), compound 7 (▴), compound 8 (▪), and compound 9 (♦). Black stapled lines at 0.05 for MIC and 30% for biofilm formation indicate activity threshold values. Two of the compounds, compound 6 and compound 8, displayed 6.3 μM activity selectively against the gram-positive S. agalactiae (FIG. 9b) as well as biofilm inhibition for S. epidermidis (FIGS. 9c) at 12.5 and 25 M, respectively. Compound 9 had 50 M activity against all gram-positive bacteria, and compound 7 showed 50 μM activity against S. aureus (FIG. 9a). No activity was observed against the gram-negative bacteria (Table 3). A decrease in activity at higher concentrations for compound 6 and compound 8 against S. agalactiae is caused by precipitation due to limited solubility.

TABLE 3
Minimum inhibitory concentration (μM) of compounds 6-9
against gram-positive bacteria. The activity was screened in
concentrations ranging from 100, 75, 50, 25, 12.5, 6.3, 3.1,
1.6 μM. (I = inactive, i.e. below the activity threshold).

| Compound | S. Epidermidis | S. Aureus | S. Agalactiae |
|---|---|---|---|
| 6 | I | I | 6.3 |
| 7 | I | 50 | I |
| 9 | 50 | 50 | 50 |
| 8 | I | I | 6.3 |
| 6-decomp | I | I | I |
| 7-decomp | I | I | I |
| 9-decomp | I | I | I |
| 8-decomp | I | I | I |

### Spectral Analysis

IR spectra were recorded on an Agilent Cary 630 FT-IR spectrophotometer equipped with an attenuated total reflectance (ATR) attachment. Samples were analysed neat on a ZnSe crystal and the absorption frequencies are given in wave numbers (cm⁻¹).

UV-vis spectra were obtained on an Agilent 8453 single-beam UV-vis spectrophotometer with a deuterium-discharge lamp for the UV range and a tungsten lamp for the visible wavelength range. Samples were analysed in an Agilent open-top UV quartz cell (10 mm, 3.0 mL) with ethanol as solvent. Wavelengths are reported in nm and molar attenuation coefficients in M⁻¹cm⁻¹.

NMR spectra were recorded on a Bruker Ascend™ 400 spectrometer (400.13 MHz for ¹H, 100.61 MHz for ¹³C, 376.46 MHz for 19F) or a Bruker Ascend™ 850 spectrometer (850.13 MHz for ¹H and 213.77 MHz for ¹³C). Coupling constants (J) are given in Hz and the multiplicity is reported as singlet (s), doublet (d), triplet (t), sextet (sxt), multiplet (m), and broad singlet (bs). The chemical shift values are reported upfield to downfield in ppm and calibration is done using the residual solvent signals for chloroform-d (¹H 7.26 ppm; ¹³C 77.16 ppm) or acetonitrile-d3 (¹H 1.94 ppm; ¹³C 1.32 ppm).²⁷ Calibration for ¹⁹F NMR is done using α,α, α-trifluorotoluene as internal standard in chloroform-d (−62.61 ppm) and acetonitrile-d3 (−63.10 ppm).

High-resolution mass spectra were obtained on a JEOL AccuTOF™ T100GC mass spectrometer operated in ESI mode. Low-resolution mass spectra were recorded on an Advion expression compact mass spectrometer (CMS) operated in ESI mode equipped with a Plate Expres® TLC plate reader for sample injection. A solution of ammonium acetate (3.0 mM) and formic acid (0.05%) in acetonitrile and water (95/5) was used as mobile phase for both positive and negative ESI modes.

Thin-layer chromatography (TLC) was carried out with silica gel (60 F₂₅₄) on aluminium sheets with solvent systems consisting of various mixtures of petroleum ether, ethyl acetate, and DCM. Staining was achieved with either exposure to UV light (254 and/or 365 nm) or a potassium permanganate stain. Flash chromatography was performed with a hand pump and 230-400 mesh silica gel.

General Synthesis Procedures

### Lewis-Acid Promoted Epoxide Ring Opening

Lithium perchlorate was dried on vacuum 1 hr and dissolved in anhydrous diethyl ether to a 5M solution. The appropriate aniline (1.0-1.3 equiv.) and epoxide (1.0 equiv.) was added (˜0.2 M) and the reaction mixture was stirred at 40° C. under an Ar atmosphere overnight. DCM and water were added, the phases were separated, and the aqueous layer was extracted with DCM (3×10 mL). The combined organic phases were evaporated on celite and subjected to silica-gel flash chromatography (Pet. Ether/DCM, 3/7), and concentration of the relevant fractions yielded the nitrophenethyl alcohols.

### Synthesis of 1-Allyl-4-Nitrobenzene

An oven-dried 25 mL round-bottom flask fitted with a condenser was charged with 1-iodo-4-nitrobenzene (1.00 g, 4.00 mmol), CsF (1.52 g, 10.0 mmol), Pd(PPh₃)₄ (0.70 g, 15 mol %), THF (20 mL), and water (2 mL). The mixture was stirred at room temperature under Ar for 30 min, followed by addition of allylboronic acid pinacol ester (1.36 mL, 7.20 mmol) in THF (8 mL). The reaction mixture was refluxed (oil bath, 95° C.) for 22 hr. After cooling to rt the product mixture was evaporated onto celite and purified by silica-gel flash chromatography (Pet. ether). Concentration of the relevant fractions (R_{f} 0.47, Pet. ether/EtOAc 8/2) yielded the allylated product (0.35 g, 52%) as a slightly yellow liquid. Spectroscopic data are in accordance with previously reported literature, see below.

TLC (Pet. Ether: EtOAc, 80:20 v:v): R_{f}=0.47.

IR (neat): vₘₐₓ 3091, 3017, 2918, 1593, 1502.

¹H NMR (400.13 MHZ, CDCl₃): δ 8.15 (d, J=8.8 Hz, 2H), 7.34 (d, J=8.8 Hz, 2H), 5.99-5.89 (m, 1H), 5.18-5.09 (m, 2H), 3.49 (d, J=6.7 Hz, 2H).

¹³C NMR (100.61 MHZ, CDCl₃): δ 147.9, 146.7, 135.6, 129.5, 123.8, 117.5, 40.0.

### Synthesis of 2-(4-Nitrobenzyl)Oxirane

An oven-dried 25 mL round-bottom flask charged with 1-allyl-4-nitrobenzene (0.35 g, 2.16 mmol) in anhydrous DCM (12 mL) was cooled (ice/water bath) and stirred for 5 min under Ar followed by addition of mCPBA (0.59 g, 2.63 mmol). The reaction mixture was stirred at ambient temperature for 2 hr, then at room temperature for 15 hr. More mCPBA (0.12 g, 0.54 mmol) was added and stirring continued for another 31 hr before quenching the reaction with 1 M aq. NaOH solution (10 mL). The phases were separated and the aq. phase was extracted with DCM (3×15 mL). The combined organic phases were washed with water (20 mL), brine (20 mL), dried (MgSO₄), filtered, and concentrated in vacuo. The product was isolated by silica-gel autoflash chromatography (Pet. ether/EtOAc/DCM, 93/2/5→40/55/5) and concentration of the relevant fractions (R_{f} 0.26, Pet. ether/DCM, 5/5) yielded the epoxide as a yellow oily liquid (0.20 g, 52%). See below for spectroscopic data.

TLC (Pet. Ether:DCM, 50:50 v:v): R_{f}=0.26.

IR (neat): vₘₐₓ 2957, 2923, 2853, 1599, 1516, 1345.

¹H NMR (400.13 MHZ, CDCl₃): δ 8.18 (d, J=8.5 Hz, 2H), 7.43 (d, J=8.5 Hz, 2H), 3.20-3.16 (m, 1H), 3.06 (dd, J=14.8 Hz, 4.2 Hz, 1H), 2.90 (dd, J=14.8 Hz, 6.4 Hz, 1H), 2.84 (dd, J=4.7 Hz, 4.0 Hz, 1H), 2.55 (dd, J=4.7 Hz, 2.6 Hz, 1H).

¹³C NMR (100.61 MHZ, CDCl₃): δ 147.1, 145.0, 130.0, 123.9, 51.7, 46.8, 38.6.

Synthesis of 1-(2,5-Dichloro-4-(1,1,2,3,3, 3-hexafluoropropoxy)phenyl)amino)-3-(4-nitrophenyl)propan-2-ol

2,5-Dichloro-4-(1,1,2,3,3,3-hexafluoropropoxy)aniline (111 mg, 0.34 mmol) and 2-(4-nitrobenzyl)oxirane (62 mg, 0.34 mmol) was reacted according to the general procedure for 19 hr to yield 6 as a white solid (87 mg, 50%, mp. 121-123° C.) and 40% recovery of 2-(4-nitrobenzyl)oxirane. See below for spectroscopic data.

TLC (DCM): R_{f} =0.36.

IR (neat): vₘₐₓ 3507 (OH), 3420, 3376 (NH), 2980, 2918, 2857, 1599.

UV/vis (EtOH): λₘₐₓ 255 nm (ε 18339 M⁻¹cm⁻¹).

¹H NMR (850.13 MHZ, CD₃CN): δ 8.14 (d, J=8.7 Hz, 2H), 7.49 (d, J=8.7 Hz, 2H), 7.33 (s, 1H), 6.81 (s, 1H), 5.55 (dsxt, J=42.8 Hz, 5.8 Hz, 1H), 5.03 (t, J=5.6 Hz, NH), 4.06-4.02 (m, 1H), 3.30 (ddd, J=13.3 Hz, 6.4 Hz, 3.9 Hz, 1H), 3.27 (d, J=4.4 Hz, OH), 3.15-3.11 (m, 1H), 2.98 (dd, J=13.8 Hz, 4.5 Hz, 1H), 2.85 (dd, J=13.8 Hz, 8.4 Hz, 1H).

¹³C NMR (213.77 MHZ, CD₃CN): δ 148.2, 147.7, 145.2, 134.5, 131.5, 127.9, 125.6, 124.3, 121.3 (qd, J=281 Hz, 25 Hz), 119.1 (td, J=271 Hz, 23 Hz), 117.7, 112.6, 85.4 (dsxt, J=198 Hz, 35 Hz), 70.8, 49.7, 41.7.

¹⁹F NMR (376.46 MHZ, CD₃CN): δ −75.6-75.7 (m, 3F), −78.4-80.4 (m, 2F), −213.3 (sxt, J=12 Hz, 1F).

HRMS: (ESI/TOF) m/z: [M+Na]⁺ Calcd for C₁₈H₁₄C₁₂F₆N₂O₄Na⁺529.01270; found 529.01286.

Synthesis of 2-Bromo-1,3-difluoro-4-nitrobenzene

2,6-Difluoro-1-bromobenzene (579 mg, 3.00 mmol) was dissolved in 95-97% sulfuric acid (4 mL) and cooled to 0° C. To this was added an ice cold mixture of 95-97% sulfuric acid (4 mL) and 65% nitric acid (5.2 mL) dropwise over 15 min. The reaction mixture was stirred at 0° C. for 1 hr, poured over ice, and extracted with DCM (3×15 mL). The combined organic layers were washed with sat. aq. NaHCO₃ solution (20 mL), dried (MgSO₄), filtered, and concentrated under reduced pressure to yield the title compound as a white crystalline solid (691 mg, 97%, mp. 52-53° C.).

TLC (Pet. Ether:EtOAc, 90:10, v:v): R_{f}=0.35.

IR (neat): vₘₐₓ 3099, 1916, 1591, 1530.

¹H NMR (400.13 MHz, CDCl₃): δ 8.12 (ddd, J=9.4 Hz, 8.0 Hz, 5.5 Hz, 1H), 7.14 (ddd, J=9.4 Hz, 7.0 Hz, 2.0 Hz, 1H).

¹³C NMR (100.61 MHz, CDCl₃): δ 163.1 (dd, J=260 Hz, 3 Hz), 154.3 (dd, J=267 Hz, 5 Hz), 134.8, 126.2 (dd, J=10 Hz, 2 Hz), 112.1 (dd, J=24 Hz, 4 Hz), 101.3 (dd, J=25 Hz, 24 Hz).

¹⁹F NMR (376.46 MHZ, CDCl₃): δ −92.0 (d, J=9.5 Hz), −104.4 (d, J=9.5 Hz).

Synthesis of S2-Allyl-1,3-difluoro-4-nitrobenzene

Aryl bromide # (870 mg, 3.66 mmol), Pd(PPh₃)₄ (634 mg, 15 mol %), and BusSnAllyl (1.53 mL, 4.39 mmol) was dissolved in anhydrous DMF (8 mL). 4 Å molecular sieves were added and the reaction mixture was stirred at 100° C. under Ar for 24 hr. The solids were filtered off through cotton with the aid of DCM (50 mL), and the volatiles were removed under reduced pressure. The title compound was isolated by silica-gel flash chromatography (Pet. Ether/EtOAc, 99/1) and concentration of the relevant fractions yielded the allylated product as a colourless liquid (194 mg, 27%). See below for spectroscopic data.

TLC (Pet. Ether: EtOAc, 95:5, v:v): R_{f}=0.32.

IR (neat): vₘₐₓ 3087, 2957, 2925, 2855, 1623, 1596.

¹H NMR (400.13 MHZ, CDCl₃): δ 8.01 (ddd, J=9.0 Hz, 8.5 Hz, 5.7 Hz, 1H), 7.04-6.99 (m, 1H), 5.95-5.85 (m, 1H), 5.13-5.08 (m, 2H), 3.51-3.48 (m, 2H).

¹³C NMR (100.61 MHz, CDCl₃): δ 164.2 (dd, J=258 Hz, 8 Hz), 155.2 (dd, J=266 Hz, 10 Hz), 134.5-134.4 (m), 132.9, 125.4 (dd, J=11 Hz, 2 Hz), 118.8 (dd, J=22 Hz, 19 Hz), 117.4, 111.7 (dd, J=25 Hz, 4 Hz), 26.8 (t, J=3 Hz). ¹⁹F NMR (376.46 MHZ, CDCl₃): δ −102.5 (d, J=14.1 Hz), −116.6 (d, J=14.1 Hz).

Synthesis of 2-(2,6-Difluoro-3-nitrobenzyl)oxirane

To a stirred solution of allylbenzene # (194 mg, 0.97 mmol) at 0° C. was added mCPBA (425 mg, 1.94 mmol). The reaction mixture was stirred for 2 hours at ° C. and 5 d at room temperature, followed by addition of a 1:1 sat. aq. NaHCO₃: 10% Na₂SO₃ solution (30 mL). The phases were separated and the aqueous layer was extracted with DCM (3×15 mL). The combined organic phases were washed with a 1:1 sat. aq. NaHCO₃:10% Na₂SO₃ solution (30 mL), sat. aq. NaHCO₃ (30 mL), water (30 mL), dried (MgSO₄), filtered and concentrated under reduced pressure to yield the allylated product as a slightly yellow oily liquid (183 mg, 88%), and was used without further purification. See below for spectroscopic data.

TLC (DCM): R_{f}=0.53.

IR (neat): vₘₐₓ 3104, 3000, 2926, 1728, 1624.

¹H NMR (400.13 MHZ, CDCl₃): δ 8.05 (ddd, J=9.2 Hz, 8.5 Hz, 5.7 Hz, 1H), 7.07-7.02 (m, 1H), 3.22-3.17 (m, 1H), 3.16-3.11 (m, 1H), 3.03-2.97 (m, 1H), 2.81-2.79 (m, 1H), 2.56 (dd, J=4.8 Hz, 2.5 Hz, 1H).

¹³C NMR (100.61 MHZ, CDCl3): δ 164.5 (dd, J=259 Hz, 8 Hz), 155.5 (dd, J=266 Hz, 9 Hz), 134.5, 126.1 (dd, J=11 Hz, 1 Hz), 115.8 (dd, J=22 Hz, 20 Hz), 111.8 (dd, J=25 Hz, 4 Hz), 50.1, 46.9, 25.8 (t, J=2 Hz).

¹⁹F NMR (376.46 MHZ, CDCl₃): δ −101.5 (d, J=13.6 Hz), -115.7 (d, J=13.6 Hz).

Synthesis of 1-((3,5-Dichloro-2-fluorophenyl)amino)-3-(2,6-difluoro-3-nitrophenyl)propan-2-ol

3,5-Dichloro-2-fluorophenylaniline (82 mg, 0.46 mmol) and 2-(2,6-difluoro-3-nitrobenzyl)oxirane (77 mg, 0.36 mmol) was reacted according to the general procedure for 20 hr to yield 9 (60 mg, 42%) as a sticky colorless oil and 7% recovery of 2-(2,6-difluoro-3-nitrobenzyl)oxirane. See below for spectroscopic data.

TLC (DCM): R_{f}=0.36.

IR (neat): vₘₐₓ 3565 (OH), 3425 (NH), 3101, 2926, 2857, 1597.

UV/vis (EtOH): λₘₐₓ 254 nm (ε16575 M⁻¹cm⁻¹).

¹H NMR (400.13 MHZ, CD₃CN): δ 8.05 (ddd, J=9.3 Hz, 8.5 Hz, 5.7 Hz, 1H), 7.14 (ddd, J=9.3 Hz, 8.5 Hz, 1.9 Hz, 1H), 6.73-6.69 (m, 2H), 4.93 (bs, NH), 4.06-3.98 (m, 1H), 3.34 (d, J=5.3 Hz, OH), 3.32 (ddd, J=13.5 Hz, 6.7 Hz, 4.0 Hz, 1H), 3.20-3.13 (m, 1H), 2.99-2.87 (m, 2H).

¹³C NMR (100.61 MHZ, CD₃CN): δ 165.5 (dd, J=256 Hz, 8 Hz), 156.2 (dd, J=263 Hz, 10 Hz), 146.8 (d, J=239 Hz), 139.9 (d, J=12 Hz), 135.5, 130.5 (d, J=4 Hz), 126.7 (dd, J=12 Hz, 2 Hz), 121.4 (d, J=16 Hz), 118.6 (dd, J=22 Hz, 19 Hz), 116.5 (d, J=1 Hz), 112.6 (dd, J=25 Hz, 4 Hz), 111.6 (d, J=4 Hz), 69.3, 49.5, 29.1.

¹⁹F NMR (376.46 MHZ, CD₃CN): δ −103.0 (d, J=13.6 Hz, 1F), −117.5 (d, J=13.6 Hz, 1F), −141.7 (s, 1F).

HRMS: (ESI/TOF) m/z: [M+H]⁺ Calcd for C₁₅H₁₂Cl₂F₃N₂O₃⁺ 395.01716; found 395.01724.

Synthesis of 1-((3,5-Dichloro-2, 4-difluorophenyl)amino)-3-(2,6-difluoro-3-nitrophenyl)propan-2-ol

3,5-Dichloro-2,4-difluorophenylaniline (65 mg, 0.33 mmol) and 2-(2,6-difluoro-3-nitrobenzyl)oxirane (71 mg, 0.33 mmol) was reacted according to the general procedure for 22 hr to yield 7 as a white solid (45 mg, 33%, mp. 110-112° C. and 46% recovery of 2-(2,6-difluoro-3-nitrobenzyl)oxirane. See below for spectroscopic data.

TLC (DCM): R_{f}=0.30.

IR (neat): vₘₐₓ 3433 (OH), 3382 (NH), 3103, 2935, 2857, 1625.

UV/vis (EtOH): λₘₐₓ 242 nm (ε16289 M⁻¹cm⁻¹).

¹H NMR (400.13 MHZ, CD₃CN): δ 8.04 (ddd, J=9.2 Hz, 8.6 Hz, 5.7 Hz, 1H), 7.13 (ddd, J=9.2 Hz, 8.5 Hz, 1.8 Hz, 1H), 6.79 (dd, J=8.6 Hz, 7.1 Hz, 1H), 4.72 (bs, NH), 4.05-3.97 (m, 1H), 3.34 (d, J=5.3 Hz, OH), 3.92 (ddd, J=13.4 Hz, 6.6 Hz, 4.1 Hz, 1H), 3.16-3.10 (m, 1H), 2.98-2.87 (m, 2H).

¹³C NMR (100.61 MHz, CD₃CN): δ 165.5 (dd, J=256 Hz, 8 Hz), 156.2 (dd, J=263 Hz, 10 Hz), 146.9 (dd, J=242 Hz, 1 Hz), 146.3 (dd, J=237 Hz, 2 Hz), 135.7 (dd, J=12 Hz, 3 Hz), 135.4 (dd, J=8 Hz, 3 Hz), 126.7 (d, J=12 Hz), 118.5 (dd, J=22 Hz, 20 Hz), 117.3 (dd, J=18 Hz, 4 Hz), 112.6 (dd, J=25 Hz, 4 Hz), 111.4-111.0 (m, 1C), 111.2 (d, J=4 Hz), 69.4, 49.7, 29.1.

¹⁹F NMR (376.46 MHz, CD₃CN): δ −103.0 (d, J=14.0 Hz, 1F), −117.5 (d, J=14.0 Hz, 1F), −133.9 (d, J=4.0 Hz, 1F), -137.0 (d, J=4.0 Hz, 1F).

HRMS: (ESI/TOF) m/z: [M+H]⁺ Calcd for C₁₅H₁₁Cl₂F₄N₂O₃⁺ 413.00774; found 413.00829.

Synthesis of 1-((2,5-Dichloro-4-(1,1,2,3,3, 3-hexafluoropropoxy)phenyl)amino)-3-(2,6-difluoro-3-nitrophenyl)propan-2-ol

2,5-Dichloro-4-(1,1,2,3,3,3-hexafluoropropoxy)aniline (151 mg, 0.46 mmol) and 2-(2,6-difluoro-3-nitrobenzyl)oxirane (100 mg, 0.46 mmol) was reacted according to the general procedure for 18 hr to yield 8 as a slightly yellow oily liquid (104 mg, 42%) and 16% recovery of 2-(2,6-difluoro-3-nitrobenzyl)oxirane. See below for spectroscopic data.

TLC (DCM): R_{f}=0.36.

IR (neat): vₘₐₓ 3550 (OH), 3411 (NH), 3103, 2937, 1624.

UV/vis (EtOH): λₘₐₓ 254 nm (ε 25248 M⁻¹cm⁻¹).

¹H NMR (400.13 MHZ, CD₃CN): δ 8.04 (ddd, J=9.2 Hz, 8.5 Hz, 5.7 Hz, 1H), 7.31 (t, J=1.0 Hz, 1H), 7.12 (ddd, J=9.2 Hz, 8.5 Hz, 1.8 Hz, 1H), 6.85 (s, 1H), 5.54 (dsxt, J=42.8 Hz, 5.9 Hz, 1H), 5.03 (t, J=5.8 Hz, NH), 4.09-4.02 (m, 1H), 3.42 (d, J=5.4 Hz, OH), 3.35 (ddd, J=13.3 Hz, 6.4 Hz, 4.1 Hz, 1H), 3.22-3.16 (m, 1H), 2.99-2.89 (m, 2H).

¹³C NMR (213.77 MHz, CD₃CN): δ 165.5 (dd, J=256 Hz, 8 Hz), 156.2 (dd, J=263 Hz, 10 Hz), 145.1, 135.5 (dd, J=8 Hz, 3 Hz), 134.6, 127.9, 126.7 (d, J=12 Hz), 125.6, 121.3 (qd, J=281 Hz, 25 Hz), 119.1 (td, J=271 Hz, 23 Hz), 118.5 (dd, J=22 Hz, 19 Hz), 117.7, 112.71, 112.67 (dd, J=25 Hz, 3 Hz), 85.4 (dsxt, J=198 Hz, 35 Hz), 69.3, 49.6, 29.2.

19F NMR (376.46 MHZ, CD₃CN): δ −75.6-75.7 (m, 3F), −78.5-80.4 (m, 2F), −102.9 (d, J=14.0 Hz, 1F), −117.4 (d, J=14.0 Hz, 1F), −213.3 (sxt, J=11.2 Hz, 1F).

HRMS: (ESI/TOF) m /z: [M+H]⁺ Calcd for C₁₈H₁₃Cl₂F₈N₂O₄⁺ 543.01191; found 543.01196.

1. A method of synthesizing a photolytically degradable compound, which method comprises the steps of
a) identifying a biologically active target molecule comprising one or more substituted aromatic group(s);
b) providing a starting material which presents at least one aromatic group identical to said one or more substituted aromatic group(s); and
c) synthesizing the photolytically degradable compound by introducing a nitro substitution in said aromatic group of the starting material;
wherein the photolytically degradable compound is defined by the general Formula I: 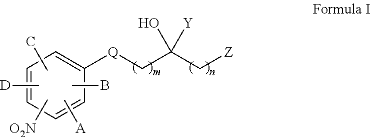
wherein
at least one of A and B and C and D is
—CN,
—CH₂NR¹R² or —[CH₂NR¹R²R³]⁺ wherein R¹, R² and R³ are each selected from a group consisting of H and branched or unbranched C₁-C₆ alkyl;
O(CH₂)ₒ(CX₂)ₚ(CH₂)_{q}(CHX)ᵣ(CH₂)ₛ(CX₂)ₜ(CH₂)ᵤ(CX₃)ᵥ(CHX₂)_{w}(CH₂X)ₓ(CH₃)_{y} where
o=0-5, p=0-5, q=0-5, r=0-5, s=0-4, t=0-4, u=0-4, v=0-1, w=0-1, x=0-1, y=0-1, provided that in each compound, only one of v, w, x, and y are
1 while the other three are 0, and
where X is F and/or Cl,
while the other three of A, B, C and D are
1) each independently selected from the group consisting of H, F, Cl, I, Br, CN, CF₃, C₁-C₆ alkyl, alkoxy with a C₁-C₆ alkyl, NH₂, NHR¹, NR¹R², and [NR¹R²R³]⁺ where R¹, R² and R³ are as defined above, and
2) can be randomly attached to the four remaining carbon atoms in the phenyl ring available for substitution;
Q is either
NH, NR⁴, [NR⁴R⁵]⁺, NOH, NOR⁴, NNH₂, NNHR⁴, [NNHR⁴R⁵]+, NNR⁴R⁵, and [NNR⁴R⁵R⁶]⁺ where R⁴, R⁵, and R⁶ are a branched or unbranched C₁-C₃ alkyl;
or O;
or S, [SR⁴]⁺, SO and SO₂;
m is 0-4, such as 1-4;
Y is selected from a group consisting of H and branched or unbranched C₁-C₆ alkyl;
n is 0-4;
Z is either
H, or
NHC(O)R⁷
wherein R⁷ is either R⁵, or monohalogenated-perhalogenated derivatives of R⁵,
wherein the halogen is F and/or Cl;
aryl; such as 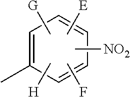
which aryl may be substituted with a nitro group, and wherein
substituents E and F and G and H are as defined above for A, B, C and D, respectively.

2. A method according to claim 1, wherein in step c), a meta-nitro substitution; a para-nitro substitution; or a meta-nitro substitution and a para-nitro substitution is introduced in said aromatic group of the starting material.

3. A method according to claim 1, wherein the Z group in Formula I is aryl; such as 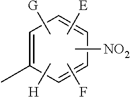
wherein
the nitro group is in meta-position or para-position to the carbon attaching the Z group to the carbon chain of Formula (I); and
substituents E and F and G and H are defined as for A, B, C and D.

4. A method according to claim 1, wherein Z is beta lactam.

5. A method according to claim 1, wherein X is F and/or Cl, such as F.

6. A method according to claim 1, wherein the starting material comprises at least one aliphatic N-, S- or O-bond and, optionally, at least one benzylic C—C bond.

7. A method according to claim 1, wherein at least one benzylic C—C bond of the photolytically degradable compound is heterolytically cleaved by exposure to light at basic pH in aqueous environment.

8. A method according to claim 7, wherein said basic pH is above about 7.

9. A method according to claim 8, wherein said basic pH equals about 11.

10. A method according to claim 1, wherein the photolytically degradable compound so synthesized presents a biological activity which is substantially equal to that of the target molecule.

11. A method according to claim 1, wherein the target molecule presents an antimicrobial activity; a pesticidal activity; or an antimicrobial activity and a pesticidal activity.

12. A method according to claim 11, wherein the target molecule is an antibacterial molecule selected from the group consisting of ciprofloxazin; chloramphenicol; and penicillin.

13. A method according to claim 11, wherein the target molecule is a pesticide selected from the group consisting of diflubenzuron; teflubenzuron, and lufenuron.

14. A photolytically degradable compound synthesized according to claim 1, which compound comprises, as a substituent, a molecule derived from an antibiotic.

15. A photolytically degradable compound according to claim 14, wherein said substituent is defined by Formula II below:

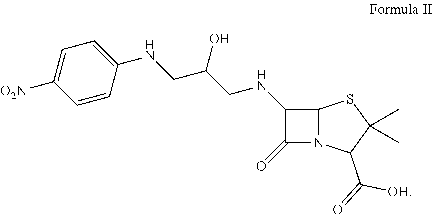 [SMILES: CC1(C)SC2C(NCC(O)CNc3ccc([N+](=O)[O-])cc3)C(=O)N2C1C(=O)O]

16. A photolytically degradable compound according to claim 14, which compound comprises, as a substituent, a molecule derived from a sea lice drug.

17. A photolytically degradable compound according to claim 14, which compound comprises, as a substituent, a molecule selected from the group consisting of diflubenzuron; teflubenzuron; and
lufenuron.

18. A method of producing purified water, in which method water, such as waste water or drinking water, which includes at least one photolytically degradable compound obtained according to the method of claim 1 is exposed to light, whereby the load of antimicrobial molecules which are photolytically degradable is reduced, which method results in water purified from antibacterial molecules; pesticidals; or antibacterials and pesticides.

19. A method according to claim 18, wherein the pH of the water is controlled to above about 7 during the light exposure.

20. A method according to claim 19, wherein said pH is controlled to about 11.

21. A method of controlling salmon lice in aquaculture, wherein a photolytically degradable compound according to claim 14 is used to treat salmon in an aquaculture.

22. A method according to claim 21, wherein said treatment is for reducing or eliminating the occurrence of salmon lice.
